SEQUENCE LISTING (21 sequences)

SEQ ID NO: 1 - DNA, 3997 bp, Artificial Sequence
  Synthetic
gacgtcaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccatgacgtctttcccgactggaaagcgggcagtgagcgcaacgcaattaatgtgagttagctcactcattaggcaccccaggctttacactttatgcttccggctcgtatgttgtgtggaattgtgagcggataacaatttcacacaggtaccatacatatggctagcatgactggtggacagcaaatgggtcgcggatccgaattcgagctccgtcgacaagcttgatatcgaattcctgcagcccgggggatcccatggtacgcgtgctagaggcatcaaataaaacgaaaggctcagtcgaaagactgggcctttcgttttatctgttgtttgtcggtgaacgctctcctgagtaggacaaatccgccgccctagacctagggtacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcccgggagatctgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtccctagtgcttggattctcaccaataaaaaacgcccggcggcaaccgagcgttctgaacaaatccagatggagttctgaggtcattactggatctatcaacaggagtccaagcgagctctcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattcc

SEQ ID NO: 2 - DNA, 4874 bp, Artificial Sequence
  Synthetic
ttccggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatgcacgaggaaatcgtcgtgcagtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccacccatgaggagtccaagcgagctctcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccgacgtcaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccatgacgtctttcccgactggaaagcgggcagtgagcgcaacgcaattaatgtgagttagctcactcattaggcaccccaggctttacactttatgcttccggctcgtatgttgtgtggaattgtgagcggataacaatttcacacaggtaccatacatatggctagcatgactggtggacagcaaatgggtcgcggatccgaattcgagctccgtcgacaagcttgatatcgaattcctgcagcccgggggatcccatggtacgcgttctagaggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgggggatcaggaccgctcccggagcgcaacccactcactacagcagagccatgtagacaacatcccctccccctttccaccgcgtcagagccccgtagcgccccctacgggctttttcatgccctgccctagcgtccaagcctcacgccgcgctcggcctctctggcggccttctggcgctcctgctgcggcgtccgctcgtgggccgcggcgggtccgcgcgccggcctcgtgcgctggcgctcgcgggcgaggtccaaggcggccgtcttcacgttctgccttgcgcagatgagatagatctagcgtggactcaaggctctcgcgaatggctcgcgttggaaactttcattgacacttgaggggcaccgcagggaaattctcgtccttgcgcagaaccggctatgtcgtgctgcgactcgagcctgcgcccttggcttgtctcgcccctctccgcgtcgctacggggcttccagcgcctttccgacgctcaccgggctggttgccctcgccgctgggctggcggccgtctatggccctgcaaacgcgccagaaacgccgtcgaagccgtgtgcgagacaccgcggccgccggcgttgtggataccacgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccagacatgatgtggacaagcctggggataagtgccctgcggtattgacacttgaggggcgcgactactgacagatgaggggcgcgatccttgacacttgaggggcagagtgatgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctggcgcgtgaccgcgcacgccgaaggggggtgcccccccttctcgaaccctcccggctaacgcgggcctcccatccccccggctgcgcccttcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggcgggcaatttttaccttgggcattcttggcatagtggtcgcgggtgccgtgctcgtgttcgggggtgaattaattccccggatcgatccgtcagcttcacgctgccgcaagcactcagggcgcaagggctgctaaaggaagcggaacacgtagaaagccagtccgcagaaacggtgctgaccccggatgaatgtcacgtactgggctatctggacaagggaaaacgcaagcgcaaacacaaacgacctagcttgcagtgggcttacatggcgatagctagactgggcggttttatggccagcaagcgaaccggaattgccagctggggcgccctctggtaaggttgggaagccctgcaaagtaaactggatggctttcttgccgccaaggatctgatggcgcaggggatcaagatcgacggatcgatccggggaa

SEQ ID NO: 3 - DNA, 6025 bp, Artificial Sequence
  Synthetic
ttccggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatgcacgaggaaatcgtcgtgcagtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccacccatgaggagtccaagcgagctctcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccgacgtcaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccatgacgtctttcccgactggaaagcgggcagtgagcgcaacgcaattaatgtgagttagctcactcattaggcaccccaggctttacactttatgcttccggctcgtatgttgtgtggaattgtgagcggataacaatttcacacaggtaccatacatatggctagcatgactggtggacagcaaatgggtcgcggatccgaattcgagctccgtcgacaagcttgatatcgaattcctgcagcccgggggatcccatggtacgcgttctagaggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgggggatcaggaccgctcccggagcgcaacccactcactacagcagagccatgtagacaacatcccctccccctttccaccgcgtcagagccccgtagcgccccctacgggctttttcatgccctgccctagcgtccaagcctcacgccgcgctcggcctctctggcggccttctggcgctcctgctgcggcgtccgctcgtgggccgcggcgggtccgcgcgccggcctcgtgcgctggcgctcgcgggcgaggtccaaggcggccgtcttcacgttctgccttgcgcagatgagatagatctagcgtggactcaaggctctcgcgaatggctcgcgttggaaactttcattgacacttgaggggcaccgcagggaaattctcgtccttgcgcagaaccggctatgtcgtgctgcgactcgagcctgcgcccttggcttgtctcgcccctctccgcgtcgctacggggcttccagcgcctttccgacgctcaccgggctggttgccctcgccgctgggctggcggccgtctatggccctgcaaacgcgccagaaacgccgtcgaagccgtgtgcgagacaccgcggccgccggcgttgtggataccacgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccagacatgatgtggacaagcctggggataagtgccctgcggtattgacacttgaggggcgcgactactgacagatgaggggcgcgatccttgacacttgaggggcagagtgatgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctggcgcgtgaccgcgcacgccgaaggggggtgcccccccttctcgaaccctcccggctaacgcgggcctcccatccccccggctgcgcccttcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggcgggcaatttttaccttgggcattcttggcatagtggtcgcgggtgccgtgctcgtgttcgggggtgaattgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccaaattccccggatcgatccgtcagcttcacgctgccgcaagcactcagggcgcaagggctgctaaaggaagcggaacacgtagaaagccagtccgcagaaacggtgctgaccccggatgaatgtcacgtactgggctatctggacaagggaaaacgcaagcgcaaacacaaacgacctagcttgcagtgggcttacatggcgatagctagactgggcggttttatggccagcaagcgaaccggaattgccagctggggcgccctctggtaaggttgggaagccctgcaaagtaaactggatggctttcttgccgccaaggatctgatggcgcaggggatcaagatcgacggatcgatccggggaa

SEQ ID NO: 4 - DNA, 7025 bp, Artificial Sequence
  Synthetic
gacgtcaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccatgacgtctttcccgactggaaagcgggcagtgagcgcaacgcaattaatgtgagttagctcactcattaggcaccccaggctttacactttatgcttccggctcgtatgttgtgtggaattgtgagcggataacaatttcacacaggtacctcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataaaagcttgatatcgaattcctgcagcccgggggatcccatggtacgcgtgctagaggcatcaaataaaacgaaaggctcagtcgaaagactgggcctttcgttttatctgttgtttgtcggtgaacgctctcctgagtaggacaaatccgccgccctagacctagggtacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcccgggagatctgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtccctagtgcttggattctcaccaataaaaaacgcccggcggcaaccgagcgttctgaacaaatccagatggagttctgaggtcattactggatctatcaacaggagtccaagcgagctctcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattcc

SEQ ID NO: 5 - DNA, 7902 bp, Artificial Sequence
  Synthetic
ttccggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatgcacgaggaaatcgtcgtgcagtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccacccatgaggagtccaagcgagctctcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccgacgtcaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccatgacgtctttcccgactggaaagcgggcagtgagcgcaacgcaattaatgtgagttagctcactcattaggcaccccaggctttacactttatgcttccggctcgtatgttgtgtggaattgtgagcggataacaatttcacacaggtacctcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataaaagcttgatatcgaattcctgcagcccgggggatcccatggtacgcgttctagaggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgggggatcaggaccgctcccggagcgcaacccactcactacagcagagccatgtagacaacatcccctccccctttccaccgcgtcagagccccgtagcgccccctacgggctttttcatgccctgccctagcgtccaagcctcacgccgcgctcggcctctctggcggccttctggcgctcctgctgcggcgtccgctcgtgggccgcggcgggtccgcgcgccggcctcgtgcgctggcgctcgcgggcgaggtccaaggcggccgtcttcacgttctgccttgcgcagatgagatagatctagcgtggactcaaggctctcgcgaatggctcgcgttggaaactttcattgacacttgaggggcaccgcagggaaattctcgtccttgcgcagaaccggctatgtcgtgctgcgactcgagcctgcgcccttggcttgtctcgcccctctccgcgtcgctacggggcttccagcgcctttccgacgctcaccgggctggttgccctcgccgctgggctggcggccgtctatggccctgcaaacgcgccagaaacgccgtcgaagccgtgtgcgagacaccgcggccgccggcgttgtggataccacgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccagacatgatgtggacaagcctggggataagtgccctgcggtattgacacttgaggggcgcgactactgacagatgaggggcgcgatccttgacacttgaggggcagagtgatgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctggcgcgtgaccgcgcacgccgaaggggggtgcccccccttctcgaaccctcccggctaacgcgggcctcccatccccccggctgcgcccttcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggcgggcaatttttaccttgggcattcttggcatagtggtcgcgggtgccgtgctcgtgttcgggggtgaattaattccccggatcgatccgtcagcttcacgctgccgcaagcactcagggcgcaagggctgctaaaggaagcggaacacgtagaaagccagtccgcagaaacggtgctgaccccggatgaatgtcacgtactgggctatctggacaagggaaaacgcaagcgcaaacacaaacgacctagcttgcagtgggcttacatggcgatagctagactgggcggttttatggccagcaagcgaaccggaattgccagctggggcgccctctggtaaggttgggaagccctgcaaagtaaactggatggctttcttgccgccaaggatctgatggcgcaggggatcaagatcgacggatcgatccggggaa

SEQ ID NO: 6 - DNA, 6030 bp, Artificial Sequence
  Synthetic
gacgtcaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccatgacgtctttcccgactggaaagcgggcagtgagcgcaacgcaattaatgtgagttagctcactcattaggcaccccaggctttacactttatgcttccggctcgtatgttgtgtggaattgtgagcggataacaatttcacacaggtaccatacatatgagtgattacgaaggtagtggtccgacagaaggtattgattatgggcactcgatggtcgtgtggccgtcaacaggactgatttcgggtggtgatgtgaaaccaggaggctcatcaggtatcgctccatccatgcctccgggatggggggattacagcccacagggtatcgcacttgtacaaagtgttctttttccgggaattattcgccggattattctggataaggaacttgaagagggagactggtcgggatggtctgtcagtgtgcatagcccttggggaaacgagaaagtttccgctgcacgaacagttcttgagaatggtttacgtggtggtttgccagaaccgtctcgcccggctgctgtttcttttgcccgtctggagcctgcttccggaaatgagcaaaaaattattcgtcttatggttacacagcaactggaacaggtaacggatattccggccagccagttaccagcagcgggtaataatgtaccggtgaaatatcgtctgatggaccttatgcagaacggtacgcaatatatggctattatcggaggtattccgatgacagtgccggtagttgatgccgttccagttccggaccggagtcgtccgggaacaaatattaaagatgtttacagtgcccctgtatcaccaaatctaccggacctggtattaagtgtgggtcagatgaatactccggttctgtctaatcccgaaattcaggaagaaggagttattgctgagacaggtaattatgttgaggctggttatacgatgtccagtaataatcatgatgtcattgttcgttttcctgaaggcagtgatgtttctccgttatatatttcaactgtagagattctggacagtaatggtctgagtcagcgtcaggaagccgaaaataaagcaaaggatgatttcagagtcaagaaagaagaagcggtagcccgggctgaagctgaaaaagcaaaagccgagttatttagtaaagcaggtgtgaaccagcctcctgtatatacacaggaaatgatggaaagggctaattcagtaatgaatgagcagggagctcttgttctgaataacactgccagttctgtacaactggcgatgaccggaactggtgtctggactgctgctggtgatattgcaggcaacataagcaagtttttcagtaatgctctggagaaagtaaccatacccgaagtgagtcccctgcttatgcggatttctcttggtgctctgtggtttcattcagaagaagctggagcaggaagtgatatcgtgccggggcgaaatctggaggcgatgttttcgctgagtgctcagatgttggctggacagggcgtggtcattgaacctggtgcgacgagtgtaaatctgcctgttcgtggacaattgataaacagtaacgggcaattagctctggatttactgaaaacagggaatgaaagtatcccggctgcggttcctgttcttaatgctgtccgtgatacagcaacaggactggataaaatcacgttaccagcagtagtaggcgcaccttcccggacgattctggttaatccggtaccacaaccttcggtgccaacagatacaggtaatcatcaacccgttccggttacaccagtgcatacaggaacggaagtaaaatcggtcgaaatgccagtaacgacgattactcctgtttctgatgttggtggactacgagattttatttactggcgtcctgatgctgctgggactggtgttgaagctgtttatgtgatgcttaatgatcctctggattcagggcgattttcgcgtaaacaacttgataaaaaatataaacatgctggtgattttggtattagtgatacaaaaaagaatcgtgaaactcttactaaattcagggatgctattgaggagcatttatcggataaggatacagtagagaaaggaacataccgaagagaaaaaggttcaaaagtttattttaatcctaatacgatgaatgtggttataattaagtcaaatggtgagttcttatctgggtggaaaataaatccagatgcggataatggtcgaatttatttagagacaggtgaactatgaggattctccatcgaattcctgcagcccgggggatcccatggtacgcgtgctagaggcatcaaataaaacgaaaggctcagtcgaaagactgggcctttcgttttatctgttgtttgtcggtgaacgctctcctgagtaggacaaatccgccgccctagacctagggtacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcccgggagatctgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtccctagtgcttggattctcaccaataaaaaacgcccggcggcaaccgagcgttctgaacaaatccagatggagttctgaggtcattactggatctatcaacaggagtccaagcgagctctcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattcc

SEQ ID NO: 7 - DNA, 5572 bp, Artificial Sequence
  Synthetic
caagagggaatttttatgagcggtggcgatggacgcggccataacacgggcgcgcatagcacaagtggtaacattaatggtggcccgaccgggcttggtgtaggtggtggtgcttctgatggctccggatggagttcggaaaataacccgtggggtggtggttccggtagcggcattcactggggtggtggttccggtcatggtaatggcggggggaatggtaattccggtggtggttcgggaacaggcggtaatctgtcagcagtagctgcgccagtggcatttggttttccggcactttccactccaggagctggcggtctggcggtcagtatttcagcgggagcattatcggcagctattgctgatattatggctgccctgaaaggaccgtttaaatttggtctttggggggtggctttatatggtgtattgccatcacaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgagcgacagaatatttcggttgtttcaggtgttccgatgagtgttccggtggttgatgcaaaacctaccgaacgtccgggtgtttttacggcatcaattccaggtgcacctgttctgaatatttcagttaataacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagccctgaccaggtaaaacaacgtcaagatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagcccagcgggcgcagacggatgtaaataataagcaggctgcatttgatgctgctgcaaaagagaagtcagatgctgatgctgcattgagttctgctatggaaagcaggaagaagaaagaagataagaaaaggagtgctgaaaataatttaaacgatgaaaagaataagcccagaaaaggttttaaagattacgggcatgattatcatccagctccgaaaactgagaatattaaagggcttggtgatcttaagcctgggataccaaaaacaccaaagcagaatggtggtggaaaacgcaagcgctggactggagataaagggcgtaagatttatgagtgggattctcagcatggtgagcttgaggggtatcgtgccagtgatggtcagcatcttggctcatttgaccctaaaacaggcaatcagttgaaaggtccagatccgaaacgaaatatcaagaaatatctttgagaggaagttatgggacgcgtgctagaggcatcaaataaaacgaaaggctcagtcgaaagactgggcctttcgttttatctgttgtttgtcggtgaacgctctcctgagtaggacaaatccgccgccctagacctagggtacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcccgggagatctgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtccctagtgcttggattctcaccaataaaaaacgcccggcggcaaccgagcgttctgaacaaatccagatggagttctgaggtcattactggatctatcaacaggagtccaagcgagctctcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccgacgtcaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccatgacgtctttcccgactggaaagcgggcagtgagcgcaacgcaattaatgtgagttagctcactcattaggcaccccaggctttacactttatgcttccggctcgtatgttgtgtggaattgtgagcggataacaatttcacacaggtac

SEQ ID NO: 8 - DNA, 4294 bp, Artificial Sequence
  Synthetic
gacgtcaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccatgacgtctttcccgactggaaagcgggcagtgagcgcaacgcaattaatgtgagttagctcactcattaggcaccccaggctttacactttatgcttccggctcgtatgttgtgtggaattgtgagcggataacaatttcacacaggtaccatggctgctggtcacagaatgtggcaaatggcagggcttaaggcccagcgggcacagacggatgtaaataataagaaggctgcatttgatgctgctgcaaaagaaaagtcagatgctgatgttgcattgagttctgcgttggagcgccgcaaacagaaagaaaataaagaaaaggacgctaaagccaaattagataaggagagtaaacggaataagccagggaaggcaacaggtaaaggaaaacctgtcaataataagtggttaaataatgcaggtaaagacttaggttctcctgttccagatcgtatagctaataaactacgtgataaggagtttaaaagtttcgatgattttcgtaagaaattctgggaagaagtgtcaaaagatcctgagttaagtaaacaatttagtaggaacaataatgatcgaatgaaggttggaaaagcgcccaagactagaacccaggatgtttcagggaagagaacttcattcgagcttcatcatgagaagccgatcagccaaaatggtggtgtctatgatatggataacatcagcgtggtaacacctaaaaggcatattgatattcaccgaggtaaataagaattcctgcagcccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcccgggagatctgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtccctagtgcttggattctcaccaataaaaaacgcccggcggcaaccgagcgttctgaacaaatccagatggagttctgaggtcattactggatctatcaacaggagtccaagcgagctctcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattcc

SEQ ID NO: 9 - DNA, 201 bp, Artificial Sequence
  Synthetic
acgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgc

SEQ ID NO: 10 - DNA, 113 bp, Artificial Sequence
  Synthetic
ggcagtgagcgcaacgcaattaatgtgagttagctcactcattaggcaccccaggctttacactttatgcttccggctcgtatgttgtgtggaattgtgagcggataacaatt

SEQ ID NO: 11 - DNA, 2149 bp, Artificial Sequence
  Synthetic
ggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcccgggagatctgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtccctagtgcttggattctca

SEQ ID NO: 12 - DNA, 2990 bp, Artificial Sequence
  Synthetic
gatcaggaccgctcccggagcgcaacccactcactacagcagagccatgtagacaacatcccctccccctttccaccgcgtcagagccccgtagcgccccctacgggctttttcatgccctgccctagcgtccaagcctcacgccgcgctcggcctctctggcggccttctggcgctcctgctgcggcgtccgctcgtgggccgcggcgggtccgcgcgccggcctcgtgcgctggcgctcgcgggcgaggtccaaggcggccgtcttcacgttctgccttgcgcagatgagatagatctagcgtggactcaaggctctcgcgaatggctcgcgttggaaactttcattgacacttgaggggcaccgcagggaaattctcgtccttgcgcagaaccggctatgtcgtgctgcgactcgagcctgcgcccttggcttgtctcgcccctctccgcgtcgctacggggcttccagcgcctttccgacgctcaccgggctggttgccctcgccgctgggctggcggccgtctatggccctgcaaacgcgccagaaacgccgtcgaagccgtgtgcgagacaccgcggccgccggcgttgtggataccacgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccagacatgatgtggacaagcctggggataagtgccctgcggtattgacacttgaggggcgcgactactgacagatgaggggcgcgatccttgacacttgaggggcagagtgatgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctggcgcgtgaccgcgcacgccgaaggggggtgcccccccttctcgaaccctcccggctaacgcgggcctcccatccccccggctgcgcccttcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggcgggcaatttttaccttgggcattcttggcatagtggtcgcgggtgccgtgctcgtgttcgggggtgaattaattccccggatcgatccgtcagcttcacgctgccgcaagcactcagggcgcaagggctgctaaaggaagcggaacacgtagaaagccagtccgcagaaacggtgctgaccccggatgaatgtcacgtactgggctatctggacaagggaaaacgcaagcgcaaacacaaacgacctagcttgcagtgggcttacatggcgatagctagactgggcggttttatggccagcaagcgaaccggaattgccagctggggcgccctctggtaaggttgggaagccctgcaaagtaaactggatggctttcttgccgccaaggatctgatggcgcaggggatcaagatcgacggatcgatccggggaattccggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatgcacgaggaaatcgtcgtgcagtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctag

SEQ ID NO: 13 - DNA, 82 bp, Artificial Sequence
  Synthetic
acgacgtcaatattgacatggtacaggccctgtactatattctcaataggtccacgactctactatatattgtgaggtaccg

SEQ ID NO: 14 - DNA, 3926 bp, Artificial Sequence
  Synthetic
gacgtcaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccatgacgtcaatattgacatggtacaggccctgtactatattctcaataggtccacgactctactatatattgtgaggtaccatacatatggctagcatgactggtggacagcaaatgggtcgcggatccgaattcgagctccgtcgacaagcttgatatcgaattcctgcagcccgggggatcccatggtacgcgtgctagaggcatcaaataaaacgaaaggctcagtcgaaagactgggcctttcgttttatctgttgtttgtcggtgaacgctctcctgagtaggacaaatccgccgccctagacctagggtacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcccgggagatctgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtccctagtgcttggattctcaccaataaaaaacgcccggcggcaaccgagcgttctgaacaaatccagatggagttctgaggtcattactggatctatcaacaggagtccaagcgagctctcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattcc

SEQ ID NO: 15 - DNA, 2175 bp, Artificial Sequence
  Synthetic
cgtgttgacataaataccactggcggtgatactgagcacaggaggtaccatggctagagatgatccgcactttaacttccgtatgcctatggaagtcagggagaaattaaaattcagggcggaggcgaatgggagatcaatgaactccgagttgttacaaatcgtccaagatgctctatcaaaaccatcgcctgtgactggatatcgcaacgatgcagaacgactcgctgatgagcagtcagagcttgttaagaagatggtgtttgatacgctgaaggatttgtacaaaaaaaccacctgacggcgggttaattggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgt

SEQ ID NO: 16 - DNA, 3595 bp, Artificial Sequence
  Synthetic
gacgtcaatattgacatggtacaggccctgtactatattctcaataggtccacgactctactatatattgtgaggtaccatacatatggctagcatgactggtggacagcaaatgggtcgcggatccgaattcgagctccgtcgacaagcttgatatcgaattcctgcagcccgggggatcccatggtacgcgtgctagaggcatcaaataaaacgaaaggctcagtcgaaagactgggcctttcgttttatctgttgtttgtcggtgaacgctctcctgagtaggacaaatccgccgccctagacctagggtacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcccgggagatctgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtccctagtgcttggattctcaccaataaaaaacgcccggcggcaaccgagcgttctgaacaaatccagatggagttctgaggtcattactggatctatcaacaggagtccaagcgagctctcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattcc

SEQ ID NO: 17 - DNA, 6623 bp, Artificial Sequence
  Synthetic
gacgtcaatattgacatggtacaggccctgtactatattctcaataggtccacgactctactatatattgtgaggtacctcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataaaagcttgatatcgaattcctgcagcccgggggatcccatggtacgcgtgctagaggcatcaaataaaacgaaaggctcagtcgaaagactgggcctttcgttttatctgttgtttgtcggtgaacgctctcctgagtaggacaaatccgccgccctagacctagggtacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcccgggagatctgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtccctagtgcttggattctcaccaataaaaaacgcccggcggcaaccgagcgttctgaacaaatccagatggagttctgaggtcattactggatctatcaacaggagtccaagcgagctctcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattcc

SEQ ID NO: 18 - DNA, 2175 bp, Artificial Sequence
  Synthetic
cgtgttgacataaataccactggcggtgatactgagcacaggaggtaccatggctagagatgatccgcactttaacttccgcatgcctatggaagtcagggagaaattaaaattcagggcggaggcgaatgggagatcaatgaactccgagttgttacaaatcgtccaagatgctctatcaaaaccatcgcctgtgactggatatcgcaacgatgcagaacgactcgctgatgagcagtcagagcttgttaagaagatggtgtttgatacgctgaaggatttgtacaaaaaaaccacctgacggcgggttaattggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgt

SEQ ID NO: 19 - DNA, 2181 bp, Artificial Sequence
  Synthetic
cgtgttgacataaataccactggcggtgatactgagcacaggaggtaccatgaaaggaatgagcaaaatgccgcagttcaatttgcgcatgcctatggaagtcagggagaaattaaaattcagggcggaggcgaatgggagatcaatgaactccgagttgttacaaatcgtccaagatgctctatcaaaaccatcgcctgtgactggatatcgcaacgatgcagaacgactcgctgatgagcagtcagagcttgttaagaagatggtgtttgatacgctgaaggatttgtacaaaaaaaccacctgacggcgggttaattggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgt

SEQ ID NO: 20 - DNA, 2589 bp, Artificial Sequence
  Synthetic
cgtgttgacataaataccactggcggtgatactgagcacaggaggtaccatggctagagatgatccgcactttaacttccgtatgcctatggaagtcagggagaaattaaaattcagggcggaggcgaatgggagatcaatgaactccgagttgttacaaatcgtccaagatgctctatcaaaaccatcgcctgtgactggatatcgcaacgatgcagaacgactcgctgatgagcagtcagagcttgttaagaagatggtgtttgatacgctgaaggatttgtacaaaaaaaccacctgacggcgggttaattggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgttggaacctcttacgtgccgatcaacgtctcattttcgccagatatcgacgtcgtgttgacataaataccactggcggtgatactgagcacaggaggtaccatgaaaggaatgagcaaaatgccgcagttcaatttgcgcatgcctatggaagtcagggagaaattaaaattcagggcggaggcgaatgggagatcaatgaactccgagttgttacaaatcgtccaagatgctctatcaaaaccatcgcctgtgactggatatcgcaacgatgcagaacgactcgctgatgagcagtcagagcttgttaagaagatggtgtttgatacgctgaaggatttgtacaaaaaaaccacctgacggcgggttaattggatcccatggtacgcgtgctagaggcatcaaataaaacgaaaggctcagtcgaaagactgggcctttcgttttatctgttgtttgtcggtgaacgctctcctgagtaggacaaatccgccgccctagacctaggcgttcggctgcggagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgt

SEQ ID NO: 21 - DNA, 5537 bp, Artificial Sequence
  Synthetic
gacgtcaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccatgacgtcaatattgacatggtacaggccctgtactatattctcaataggtccacgactctactatatattgtgaggtaccaagagggaatttttatgagcggtggcgatggacgcggccataacacgggcgcgcatagcacaagtggtaacattaatggtggcccgaccgggcttggtgtaggtggtggtgcttctgatggctccggatggagttcggaaaataacccgtggggtggtggttccggtagcggcattcactggggtggtggttccggtcatggtaatggcggggggaatggtaattccggtggtggttcgggaacaggcggtaatctgtcagcagtagctgcgccagtggcatttggttttccggcactttccactccaggagctggcggtctggcggtcagtatttcagcgggagcattatcggcagctattgctgatattatggctgccctgaaaggaccgtttaaatttggtctttggggggtggctttatatggtgtattgccatcacaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgagcgacagaatatttcggttgtttcaggtgttccgatgagtgttccggtggttgatgcaaaacctaccgaacgtccgggtgtttttacggcatcaattccaggtgcacctgttctgaatatttcagttaataacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagccctgaccaggtaaaacaacgtcaagatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagcccagcgggcgcagacggatgtaaataataagcaggctgcatttgatgctgctgcaaaagagaagtcagatgctgatgctgcattgagttctgctatggaaagcaggaagaagaaagaagataagaaaaggagtgctgaaaataatttaaacgatgaaaagaataagcccagaaaaggttttaaagattacgggcatgattatcatccagctccgaaaactgagaatattaaagggcttggtgatcttaagcctgggataccaaaaacaccaaagcagaatggtggtggaaaacgcaagcgctggactggagataaagggcgtaagatttatgagtgggattctcagcatggtgagcttgaggggtatcgtgccagtgatggtcagcatcttggctcatttgaccctaaaacaggcaatcagttgaaaggtccagatccgaaacgaaatatcaagaaatatctttgagaggaagttatgggacgcgtgaattcctgcagcccgggggatcccatggtacgcgtgctagaggcatcaaataaaacgaaaggctcagtcgaaagactgggcctttcgttttatctgttgtttgtcggtgaacgctctcctgagtaggacaaatccgccgccctagacctagggtacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcccgggagatctgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtccctagtgcttggattctcaccaataaaaaacgcccggcggcaaccgagcgttctgaacaaatccagatggagttctgaggtcattactggatctatcaacaggagtccaagcgagctctcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattcc

(19) United States
(12) Patent Application Publication
(10) Pub. No.: US 2005/0181395 A1
(43) Pub. Date: Aug. 18, 2005

(54) Systems for tightly regulated gene expression

(75) Inventors: Larry Anthony, Madison, WI (US); Marcin Filutowicz, Madison, WI (US); Hideki Suzuki, Madison, WI (US)

Correspondence Address: MEDLEN & CARROLL, LLP;Suite 350, 101 Howard Street, San Francisco, CA, 94105, US

(73) Assignee: ConjuGon, Inc., Madison, WI (US)

(21) Appl. No.: 11/010,599

(22) Filed: Dec. 13, 2004

Related U.S. Application Data
(60) Provisional application No. 60/529,255, filed on Dec. 12, 2003.

(51) Int. Cl. C12Q 1/68; C07H 21/04; C12N 1/21; C12P 21/06; C12N 15/74

(52) U.S. Cl. 435/6; 435/69.1; 435/488; 435/320.1; 435/252.33; 536/23.2

(57) ABSTRACT

The present invention relates to bacterial expression vectors. In particular, the present invention provides tightly-regulated bacterial expression vectors designed for the cloning and expression of toxic proteins, RNA, and metabolites in vivo. The present invention thus provides methods of expressing protein and RNAs that were previously not able to be expressed.

This application claims priority to Provisional Patent Application Ser. No. 60/529,255, filed Dec. 12, 2003, which is incorporated herein by reference in its entirety.

### FIELD OF THE INVENTION

The present invention relates to bacterial expression vectors. In particular, the present invention provides tightly-regulated bacterial expression vectors designed for the cloning and expression of toxic proteins, RNA, and metabolites in vivo.

### BACKGROUND OF THE INVENTION

Although many prokaryotic expression systems have been developed for expression of recombinant proteins, most gene expression systems in gram-negative bacteria such as Escherichia coli have relied exclusively on a limited set of bacterial promoters. The most widely used bacterial promoters have included the lactose (lac) (Yanisch-Perron et al. Gene 33: 103-109 {1985}), tryptophan (trp) (Tacon et al. Mol. Gen. Genet. 177:427-38 {1980}), and hybrid derivatives such as the tac (deBoer et al. Proc. Natl. Acad. Sci. U.S.A. 80:21-25 {1983}) and trc (Brosius. Gene 27: 161-172 {1984}; Amanna and Brosius. Gene 40: 183-190 {1985}) promoters. Other expression systems include use of the phage lambda promoters (PL and PR) (Bernard et al. Gene 5:59-76 {1979}; Elvin et al. Gene 37: 123-126 {1990}), the phage T7 promoter (Studier et al. J. Mol. Biol. 189:113-130 {1986}), and phage T5 promoter (Bujard et al. Methods Enzymol. 155:416-433 {1987}). While these systems are commonly used and contain many desirable features, these expression systems are subject to leaky expression from the promoters, which can prohibit cloning of extremely toxic proteins, RNA, or enzymes producing toxic metabolites.

There are several existing methods of regulating expression from these common expression systems. Bacterial promoters are usually regulated by the binding of repressor proteins to specific DNA operator sequences located within the promoter. Expression systems have typically utilized the lacI, λcI, cro, or tetracycline repressor proteins. Phage T7 expression systems utilize the regulated expression of T7 RNA polymerase to drive expression of a cloned gene that resides on a bacterial plasmid. Phage T5 expression systems control gene expression by combining the use of repressor proteins with a phage T5 promoter and high levels of repressor protein.

While these bacterial and phage systems offer the ability to express a gene at high levels of expression, they often suffer from unwanted background expression of the gene. This “leaky” expression under repressed conditions is primarily due to three factors. First, bacterial repressor proteins do not bind to DNA operator sites and prevent gene transcription with 100% efficiency. The affinity of repressor and operator as well as the relative abundance of repressor protein can lead to significant levels of background expression. Second, the majority of commercially available expression systems utilize plasmid constructs of mid to high copy number to facilitate DNA construction and molecular biology techniques, however compromising regulation of the cloned insert. When the insert is on such a plasmid, unwanted background expression of the insert can be multiplied by the plasmid copy number, leading to increased amounts of background gene expression. Third, commercially available systems are subject to read-through transcription of the cloned insert from other strong promoters located on the plasmid DNA.

The incomplete repression of promoter constructs combined with the effects of high copy number plasmids and transcriptional read-through presents a major problem when cloning genes that encode products lethal to the bacterial host. Because many of these toxic proteins are lethal at very low amounts (1-10 molecules), any background expression will prevent cloning of these genes.

Thus, the art is in need of expression constructs where the promoter tightly regulates gene expression during culture propagation when gene expression is undesirable and lethal to the bacterial host. It would also be advantageous for this expression system to replicate and thus be useful in a wide range of Gram positive and Gram negative bacteria.

### SUMMARY OF THE INVENTION

The present invention relates to bacterial expression vectors. In particular, the present invention provides tightly-regulated bacterial expression vectors designed for the cloning and expression of toxic proteins, RNA, and metabolites in vivo.

For example, in some embodiments, the present invention provides a composition comprising a vector comprising transcription terminators and a low copy number origin of replication (e.g., the vectors described by SEQ ID NOs: 1, 2, 3 and 14). The present invention is not limited to particular transcription terminators. In some preferred embodiments, the transcription terminators are rrnB ribosomal terminators T1 and T2 (e.g., those described by SEQ ID NO:9). The present invention is also not limited to a particular low copy number origin of replication. In some preferred embodiments, the low number copy origin of replication is a low copy number modified pSC101 origin of replication (e.g., as described by SEQ ID NO:10) or a RK2 origin of replication (e.g., as described by SEQ ID NO:11). In other embodiments, the low copy number origin of replication is a wildtype pSC101 origin of replication, a plSa origin of replication, or a pACYC origin of replication.

In some embodiments, the vector further comprises a promoter. The present invention is not limited to a particular promoter. In some embodiments, the promoter comprises an operator, so as to be a promoter/operator. In some preferred embodiments, the promoter/operator is the lactose promoter/operator. In other preferred embodiments, the promoter/operator is a hybrid mutant Mnt-Arc promoter operator (e.g., as described by SEQ ID NO:13). In other embodiments, the promoter is a PBAD, T7, or T5 promoter. In some preferred embodiments, the vector further comprises a multiple cloning site. In some embodiments, the vector further comprises a selectable marker.

In some embodiments, the vector comprises a plurality of terminator-promoter-gene segments or “cassettes”, e.g., for use when expressing different subunits of a toxin, or expressing multiple toxin genes on the same vector. In some embodiments, each cassette in said plurality of cassettes contains the same terminator-promoter region. In some preferred embodiments, at least one cassette of said plurality of cassettes comprises different terminators or different promoters. In some particularly preferred embodiments, each cassette of said plurality of cassettes comprises different terminators and different promoters.

In some embodiments, the vector further comprises a nucleic acid sequence encoding a protein or RNA of interest. In some embodiments, the protein or RNA is a toxic protein or toxic RNA. In other embodiments, the protein has a toxic metabolite.

In further embodiments, the present invention provides a composition comprising a hybrid mutant Mnt-Arc promoter operator nucleic acid (e.g., the hybrid mutant Mnt-Arc promoter operator nucleic acid having the nucleic acid sequence of SEQ ID NO:13). In some embodiments, the present invention provides a vector comprising the nucleic acid (e.g., the vector of SEQ ID NO:14). In some embodiments, the vector further comprises transcription terminators and a low copy number origin of replication. The present invention is not limited to particular transcription terminators. In some preferred embodiments, the transcription terminators are rrnB ribosomal terminators T1 and T2 (e.g., those described by SEQ ID NO:9). The present invention is also not limited to a particular low copy number origin of replication. In some preferred embodiments, the low number copy origin of replication is a low copy number modified pSC101 origin of replication (e.g., as described by SEQ ID NO:10) or a RK2 origin of replication (e.g., as described by SEQ ID NO:11). In other embodiments, the low copy number origin of replication is a wildtype pSC101 origin of replication, a p 15a origin of replication, or a pACYC origin of replication.

In some embodiments, the vector comprises a plurality of terminator-promoter-gene segments or “cassettes”, e.g., for use when expressing different subunits of a toxin, or expressing multiple toxin genes on the same vector. In some embodiments, each cassette in said plurality of cassettes contains the same terminator-promoter region. In some preferred embodiments, at least one cassette of said plurality of cassettes comprises different terminators or different promoters. In some particularly preferred embodiments, each cassette of said plurality of cassettes comprises different terminators and different promoters.

In some embodiments, the vector further comprises a nucleic acid sequence encoding a protein or RNA of interest. In some embodiments, the protein or RNA is a toxic protein or toxic RNA. In other embodiments, the protein has a toxic metabolite.

The present invention further provides a method, comprising providing a gene of interest inserted into a vector comprising transcription terminators and a low copy number origin of replication; and expressing the gene of interest in a bacterial host. In some embodiments, the gene of interest encodes a toxic protein or RNA. In other embodiments, the gene of interest encodes a protein with a toxic metabolite. In preferred embodiments, the gene of interest is maintained in the vector under growth conditions and the protein (e.g., a toxic protein) accumulates in the bacterial host.

The present invention is not limited to particular transcription terminators. In some preferred embodiment, the transcription terminators comprise rrnB ribosomal terminators T1 and T2 (e.g., those described by SEQ ID NO:9). In some embodiments, the transcription terminators comprise bacteriophage lambda terminators. In yet other embodiments, the terminators comprise E. coli trp gene terminators. The present invention is also not limited to a particular low copy number origin of replication. In some preferred embodiments, the low copy number origin of replication is a low copy number modified pSC101 origin of replication (e.g., as described by SEQ ID NO:10) or a RK2 origin of replication (e.g., as described by SEQ ID NO:11). In other embodiments, the low copy number origin of replication is a wildtype pSC101 origin of replication, a p15a origin of replication, or a pACYC origin of replication.

In some embodiments, the vector further comprises a promoter. The present invention is not limited to a particular promoter. In some preferred embodiments, the promoter is the lactose promoter/operator. In other preferred embodiments, the promoter/operator is a hybrid mutant Mnt-Arc promoter operator (e.g., as described by SEQ ID NO:13). In other embodiments, the promoter is a PBAD, T7, or T5 promoters. In some preferred embodiments, the vector further comprises a multiple cloning site. In some embodiments, the vector further comprises a selectable marker. In some embodiments, the vector has the nucleic acid sequence of SEQ ID NOs: 1, 2, 3 or 14. In some embodiments, the bacterial host is a gram negative bacterium (e.g., E. coli).

The present invention further provides a method, comprising providing a gene of interest inserted into a vector (e.g., the vector having the nucleic acid sequence of SEQ ID NO:14) comprising a hybrid mutant Mnt-Arc promoter operator nucleic acid (e.g., the hybrid mutant Mnt-Arc promoter operator nucleic acid having the nucleic acid sequence of SEQ ID NO:13); and expressing the gene of interest in a bacterial host. In some embodiments, the gene of interest encodes a toxic protein or RNA. In other embodiments, the gene of interest encodes a protein with a toxic metabolite. In preferred embodiments, the gene of interest is maintained in the vector under growth conditions and the protein (e.g., a toxic protein) accumulates in the bacterial host.

In some embodiments of the method, the vector further comprises transcription terminators and a low copy number origin of replication. The present invention is not limited to particular transcription terminators. In some preferred embodiment, the transcription terminators comprise rrnB ribosomal terminators T1 and T2 (e.g., those described by SEQ ID NO:9). In some embodiments, the transcription terminators comprise bacteriophage lambda terminators. In yet other embodiments, the terminators comprise E. coli trp gene terminators. The present invention is also not limited to a particular low copy number origin of replication. In some preferred embodiments, the low copy number origin of replication is a low copy number modified pSC101 origin of replication (e.g., as described by SEQ ID NO:10) or a RK2 origin of replication (e.g., as described by SEQ ID NO:11). In other embodiments, the low copy number origin of replication is a wildtype pSC101 origin of replication, a p15a origin of replication, or a pACYC origin of replication. In some embodiments, the method further provides a hybrid mutant Mnt-Arc repressor protein.

In additional embodiments, the present invention provides a kit comprising a vector comprising a hybrid mutant Mnt-Arc promoter nucleic acid; and a hybrid mutant Mnt-Arc repressor protein. In some embodiments, the hybrid mutant Mnt-Arc promoter nucleic acid has the nucleic acid sequence of SEQ ID NO:13. In certain embodiments, the kit further comprises instructions for using said kit for expressing a gene of interest encoding a toxic protein or RNA.

### DESCRIPTION OF THE FIGURES

FIG. 1 shows a schematic of a portion of an exemplary vector of the present invention.

FIG. 2 shows a map of plasmid pCON3-86B.

FIG. 3 shows a map of plasmid pCON7-74.

FIG. 4 shows a map of plasmid pCON7-71.

FIG. 5 shows a map of plasmid pCON5-25.

FIG. 6 shows a map of plasmid pCON7-77.

FIG. 7 shows a map of plasmid pCON7-58.

FIG. 8 shows a map of plasmid pCON4-42.

FIG. 9 shows a map of plasmid pCON7-11.

FIG. 10 shows the results of gene expression assays utilizing vectors of the present invention.

FIGS. 11A-11I show nucleic acid sequences of exemplary vectors and vector components of the present invention.

FIG. 12 shows a schematic of the wildtype Mnt operator, wildtype Arc operator, and the hybrid promoter/operator of the present invention.

FIG. 13 shows a map of one exemplary expression vector of the present invention (pCON12-68A).

FIG. 14 shows the nucleic acid sequence (SEQ ID NO:13) of the hybrid Mnt-Arc promoter of the present invention.

FIG. 15 shows promoter activities of some vectors of the present invention using b-galactosidase assays.

FIG. 16 shows a map of plasmid pCON9-53.

FIG. 17 shows a map of plasmid pCON12-25E.

FIG. 18 shows a map of plasmid pCON12-29E.

FIG. 19 shows a map of plasmid pCON12-35.

FIG. 20 shows a map of plasmid pCON12-44.

FIG. 21 shows a map of plasmid pCON12-55.

FIG. 22 shows a map of plasmid pCON12-68A.

FIG. 23 shows a map of plasmid pCON12-82.

FIGS. 24A-24H show nucleic acid sequences of exemplary vectors and vector components of the present invention.

### DEFINITIONS

To facilitate an understanding of the invention, a number of terms are defined below.

As used herein, the term “nucleotide” refers to a monomeric unit of nucleic acid (e.g. DNA or RNA) consisting of a sugar moiety (pentose), a phosphate group, and a nitrogenous heterocyclic base. The base is linked to the sugar moiety via the glycosidic carbon (1′ carbon of the pentose) and that combination of base and sugar is called a nucleoside. When the nucleoside contains a phosphate group bonded to the 3′ or 5′ position of the pentose it is referred to as a nucleotide. A sequence of operatively linked nucleotides is typically referred to herein as a “base sequence” or “nucleotide sequence” or “nucleic acid sequence,” and is represented herein by a formula whose left to right orientation is in the conventional direction of 5′-terminus to 3′-terminus.

As used herein, the term “base pair” refers to the hydrogen bonded nucleotides of, for example, adenine (A) with thymine (T), or of cytosine (C) with guanine (G) in a double stranded DNA molecule. In RNA, uracil (U) is substituted for thymine. This term base pair is also used generally as a unit of measure for DNA length. Base pairs are said to be “complementary” when their component bases pair up normally by hydrogen bonding, such as when a DNA or RNA molecule adopts a double stranded configuration.

As used herein, the terms “nucleic acid” and “nucleic acid molecule” refer to any nucleic acid containing molecule including, but not limited to DNA or RNA. The term encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5 carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

DNA molecules are said to have “5′ ends” and “3′ ends” because mononucleotides are joined to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. A double stranded nucleic acid molecule may also be said to have a 5′ and 3′ end, wherein the “5′” refers to the end containing the accepted beginning of the particular region, gene, or structure. A nucleic acid sequence, even if internal to a larger oligonucleotide, may also be said to have 5′ and 3′ ends (these ends are not ‘free’). In such a case, the 5′ and 3′ ends of the internal nucleic acid sequence refer to the 5′ and 3′ ends that said fragment would have were it isolated from the larger oligonucleotide. In either a linear or circular DNA molecule, discrete elements may be referred to as being “upstream” or 5′ of the “downstream” or 3′ elements. Ends are said to “compatible” if a) they are both blunt or contain complementary single strand extensions (such as that created after digestion with a restriction endonuclease) and b) at least one of the ends contains a 5′ phosphate group. Compatible ends are therefore capable of being ligated by a double stranded DNA ligase (e.g. T4 DNA ligase) under standard conditions.

As used herein, the term “hybridization” or “annealing” refers to the pairing of complementary nucleotide sequences (strands of nucleic acid) to form a duplex, heteroduplex, or complex containing more than two single-stranded nucleic acids, by establishing hydrogen bonds between/among complementary base pairs. Hybridization is a specific, i.e. non-random, interaction between/among complementary polynucleotides that can be competitively inhibited.

As used herein, the term “circular vector” refers to a closed circular nucleic acid sequence capable of replicating in a host.

As used herein, the terms “vector” or “plasmid” is used in reference to extra-chromosomal nucleic acid molecules capable of replication in a cell and to which an insert sequence can be operatively linked so as to bring about replication of the insert sequence. Examples include, but are not limited to, circular DNA molecules such as plasmids constructs, phage constructs, cosmid vectors, etc., as well as linear nucleic acid constructs (e.g., lambda phage constructs, bacterial artificial chromosomes (BACs), etc.). A vector may include expression signals such as a promoter and/or a terminator, a selectable marker such as a gene conferring resistance to an antibiotic, and one or more restriction sites into which insert sequences can be cloned.

As used herein, the terms “polylinker” or “multiple cloning site” refer to a cluster of restriction enzyme sites on a nucleic acid construct, which are utilized for the insertion, and/or excision of nucleic acid sequences.

As used herein, the term “host cell” refers to any cell that can be transformed with heterologous DNA (such as a vector). Examples of host cells include, but are not limited to, E. coli strains that contain the F or F′ factor (e.g., DH5αF or DH5αF′) or E. coli strains that lack the F or F′ factor (e.g. DH10B).

The terms “nucleic acid molecule encoding,” “DNA sequence encoding,” and “DNA encoding” refer to a sequence of nucleotides that, upon transcription into RNA and subsequent translation into protein, would lead to the synthesis of a given peptide. These terms also refer to a sequence of nucleotides that upon transcription into RNA produce RNA having a non-coding function (e.g., a ribosomal or transfer RNA). Such transcription and translation may actually occur in vitro or in vivo, or it may be strictly theoretical, based on the standard genetic code.

The term “gene” refers to a nucleic acid (e.g., DNA) sequence that comprises coding sequences necessary for the production of an RNA having a non-coding function (e.g., a ribosomal or transfer RNA), a polypeptide or a precursor. The RNA or polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the sequences located adjacent to the coding region on both the 5′ and 3′ ends for a distance of about 1 kb or more on either end, such that the gene is capable of being transcribed into a full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

The term “expression” as used herein is intended to mean the transcription (e.g. from a gene) and, in some cases, translation to gene product. In the process of expression, a DNA chain coding for the sequence of gene product is first transcribed to a complementary RNA, which is often a messenger RNA, and, in some cases, the transcribed messenger RNA is then translated into the gene protein product.

The terms “in operable combination” or “operably linked” as used herein refer to the linkage of nucleic acid sequences in such a manner that a nucleic acid molecule capable of directing the synthesis of a desired protein molecule is produced. When a promoter sequence is operably linked to sequences encoding a protein, the promoter directs the expression of mRNA that can be translated to produce a functional form of the encoded protein. The term also refers to the linkage of amino acid sequences in such a manner that a functional protein is produced.

As used herein, the term “toxic protein” refers to a protein that results in cell death or inhibits cell growth when expressed in a host cell.

As used herein, the term “toxic RNA” refers to an RNA that results in cell death or inhibits cell growth when expressed in a host cell.

As used herein, the term “toxic metabolite” refers to a metabolite of a protein that results in cell death or inhibits cell growth when the protein is expressed in a host cell.

The term “prokaryotic termination sequence,” “transcriptional terminator,” or “terminator” refers to a nucleic acid sequence, recognized by an RNA polymerase, that results in the termination of transcription. Prokaryotic termination sequences commonly comprise a GC-rich region that has a twofold symmetry followed by an AT-rich sequence. A commonly used prokaryotic termination sequence is the T7 termination sequence. A variety of termination sequences are known in the art and may be employed in the nucleic acid constructs of the present invention, including the T_{INT}, T_{L1}, T_{L2}, T_{L3}, T_{R1}, T_{R2}, T_{6S} termination signals derived from the bacteriophage lambda, ribosomal termination signals such as rrnB terminators T1 and T2 (rrnBTlT2) and termination signals derived from bacterial genes such as the trp gene of E. coli.

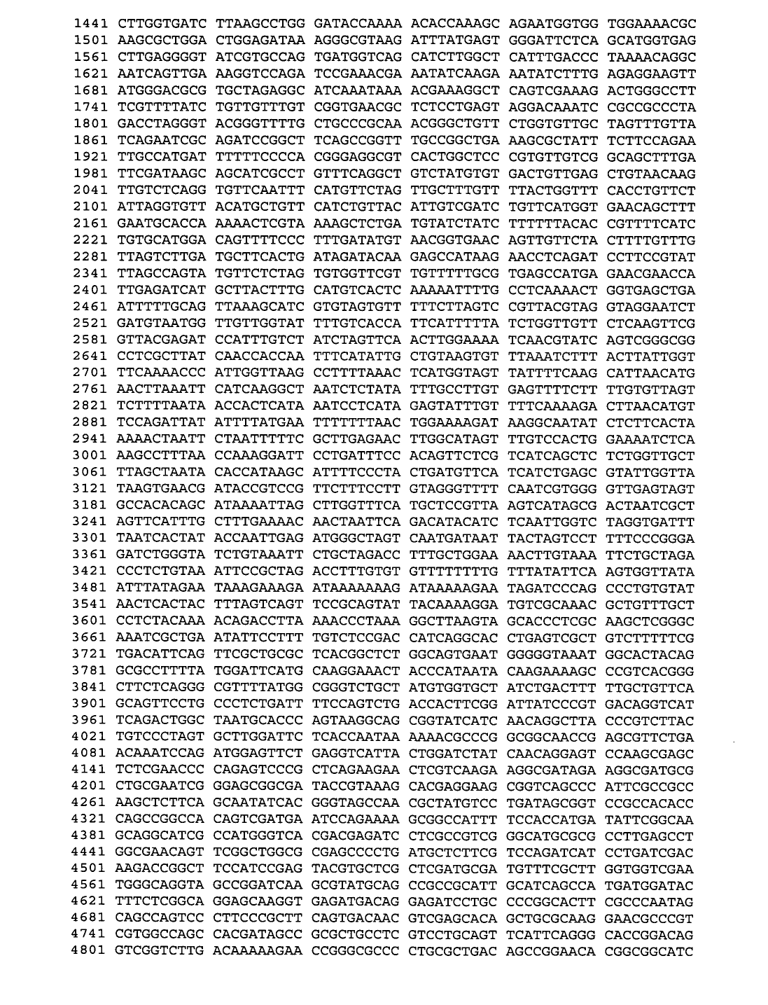

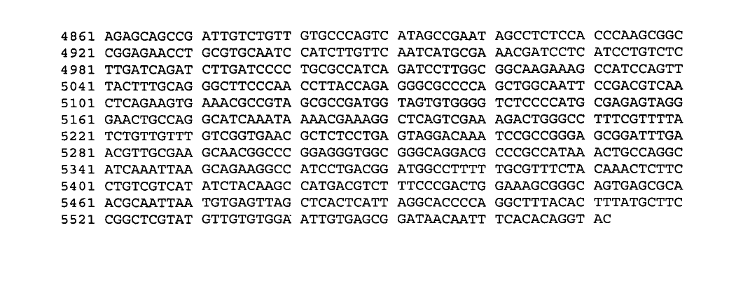

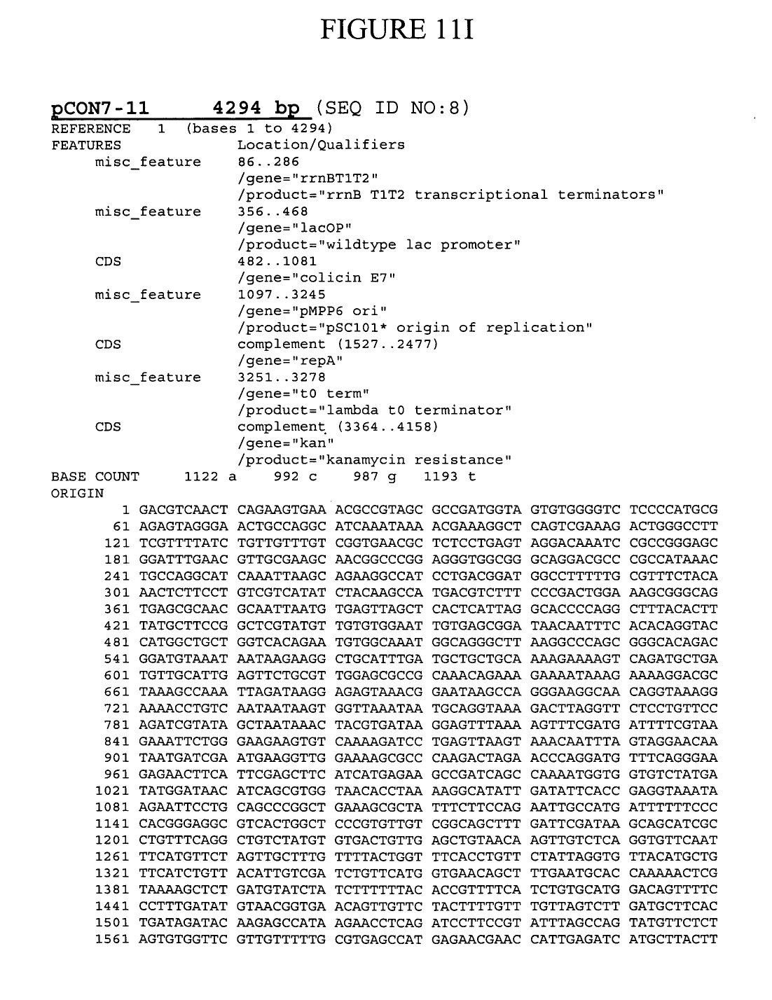

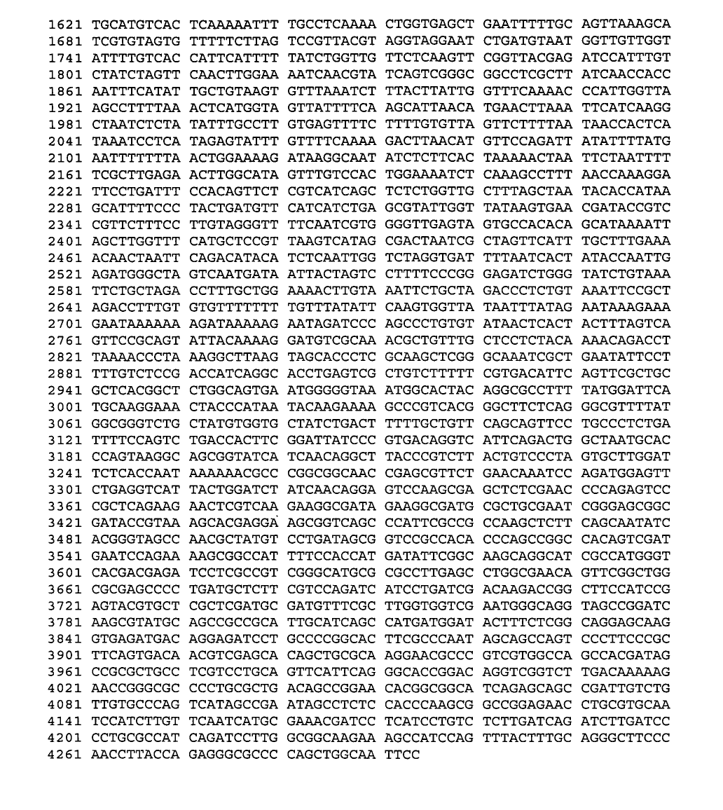

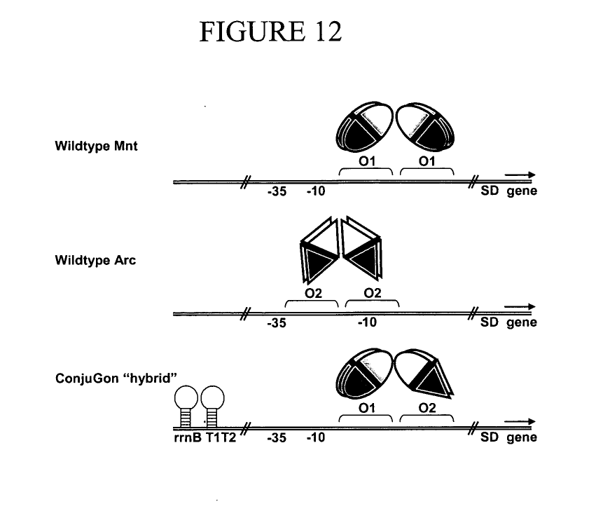

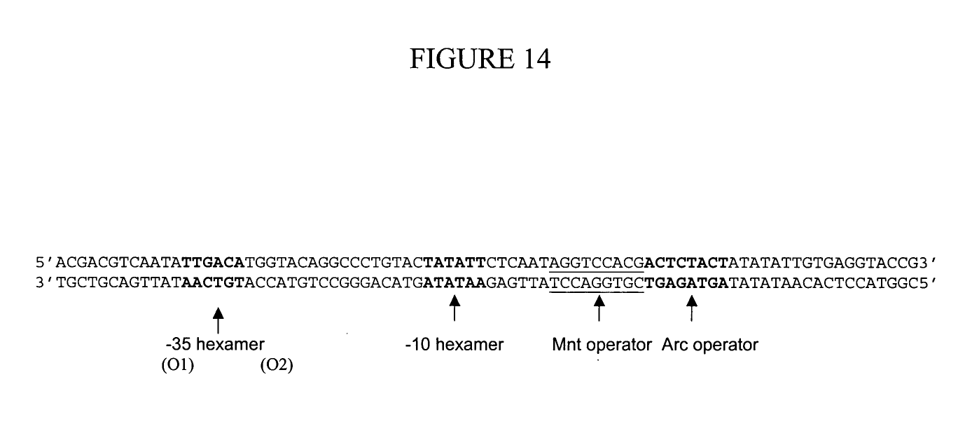

As used herein, the term “hybrid mutant Mnt-Arc promoter operator” refers to a promoter sequence (a “hybrid mutant Mnt-Arc promoter”) that is recognized by a Mnt-Arc homodimer. In some embodiments, the promoter sequence comprises one Arc operator binding sequence (O₂) and one Mnt operator binding sequence (01). A schematic of one exemplary hybrid mutant Mnt-Arc promoter operator system is shown in FIG. 12). In some preferred embodiments, the hybrid mutant Mnt-Arc promoter has the nucleic acid sequence of SEQ ID NO:13 (shown in FIG. 14).

As used herein, the term “replicable vector” means a vector that is capable of replicating in a host cell.

The term “expression vector” as used herein refers to a recombinant DNA molecule containing a desired coding sequence and appropriate nucleic acid sequences necessary for expression of the operably linked coding sequence (e.g. insert sequence that codes for a product) in a particular host organism. Nucleic acid sequences necessary for expression in prokaryotes usually include a promoter, an operator (optional), and a ribosome binding site, often along with other sequences.

As used herein, the terms “restriction endonucleases” and “restriction enzymes” refer to enzymes (e.g. bacterial), each of which cut double-stranded DNA at or near a specific nucleotide sequence. Examples include, but are not limited to, AvaII, BamHI, EcoRI, HindIII, HincII, NcoI, SmaI, and RsaI.

As used herein, the term “restriction” refers to cleavage of DNA by a restriction enzyme at its restriction site.

As used herein, the term “restriction site” refers to a particular DNA sequence recognized by its cognate restriction endonuclease.

As used herein, the term “purified” or “to purify” refers to the removal of contaminants from a sample. For example, plasmids are grown in bacterial host cells and the plasmids are purified by the removal of host cell proteins, bacterial genomic DNA, and other contaminants. Thus the percent of plasmid DNA is thereby increased in the sample. In the case of nucleic acid sequences, “purify” refers to isolation of the individual nucleic acid sequences from each other.

As used herein, the term “PCR” refers to the polymerase chain reaction method of enzymatically amplifying a region of DNA. This exponential amplification procedure is based on repeated cycles of denaturation, oligonucleotide primer annealing, and primer extension by a DNA polymerizing agent such as a thermostable DNA polymerase (e.g. the Taq or Tfl DNA polymerase enzymes isolated from Thermus aquaticus or Thermus flavus, respectively).

As used herein, the terms “complementary” or “complementarity” are used in reference to polynucleotides (i.e., a sequence of nucleotides) related by the base-pairing rules. For example, for the sequence “5′-A-G-T-3′,” is complementary to the sequence “3′-T-C-A-5′” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

As used herein, the term “oligonucleotide,” refers to a short length of single-stranded polynucleotide chain. Oligonucleotides are typically less than 100 residues long (e.g., between 15 and 50), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

The term “transformation” or “transfection” as used herein refers to the introduction of foreign DNA into cells (e.g. prokaryotic cells). Transformation may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

### DESCRIPTION OF THE INVENTION

In some embodiments, the present invention provides a bacterial expression system capable of extremely tight regulation of cloned genes. In some embodiments, this system utilizes the combination of rrnB T1T2 transcriptional terminators upstream of the wildtype lactose promoter with either the very low copy modified-pSC101 origin of replication or low copy broad-host range RK2 origin of replication. The combination of these two elements results in extremely tight regulation of the expression of the cloned gene, which allows the cloning of genes encoding extremely toxic proteins (e.g., colicin D, colicin E3, and colicin E7), which are unable to be cloned into other expression systems without the respective immunity proteins.

Most commercial expression systems (e.g., pET vectors, PBAD vectors, etc.) contain very strong promoters coupled with medium-to-high copy origins of replication, which invariably lead to “leaky” expression of the cloned gene. In addition, protein expression vectors usually have very strong bacterial (PTRC, PBAD) or phage (T7, T5) promoters that are unable to be completely repressed in the absence of inducer. Researchers often experience problems cloning toxic genes into these types of expression vectors. These origins of replication are also narrow host-range and cannot replicate in all Gram negative bacteria.

The vectors of the present invention solve many of the problems of the prior art. The combination of upstream transcriptional terminators with the low copy modified origins of replication allows the stable cloning and expression of extremely toxic proteins.

I. Vectors

In some embodiments, the present invention provides vectors for the expression of extremely toxic proteins. In preferred embodiments, the vectors of the present invention (See Table 1 in the Experimental Section for descriptions of exemplary vectors) comprise rrnBT1T2 transcription terminators (e.g., the rrnBT1T2 terminator having the sequence of SEQ ID NO:9) upstream of a strong bacterial promoter. The present invention is not limited to the use of the rrnBT1T2 transcription terminators. Other known transcription terminators may be utilized.

In some embodiments, the lactose promoter and operator (e.g., those described by SEQ ID NO:10) are utilized. In some embodiments, the LACIQ repressor protein is included on the vector. In other embodiments, it is provided on a separate vector, F′ element, or chromosome. The present invention in not limited to the use of lactose promoter and operator. Other suitable promoters may be utilized, including, but not limited to, tetracycline, PBAD, T7, and T5 promoters.

In some embodiments, the present invention provides vectors comprising a novel hybrid promoter/operator system. The hybrid promoter/operator utilizes the Arc and Mnt repressor proteins from Salmonella bacteriophage P22 as basic scaffolds.

The Arc and Mnt repressor proteins are small transcriptional regulatory proteins with structural similarity. Both Arc and Mnt proteins contain two functional domains—a dimeric N-terminal domain that binds operator DNA and a C-terminal coiled-coil domain that mediates protein tetramerization, which is essential for function (Knight and Sauer. Proc. Natl. Acad. Sci. USA 86:797-801 {1989}) (shown in FIG. 12). Tetramerization of Arc and Mnt provide cooperative interactions that increase both the binding affinity and specificity for the operator sites (Berggrun and Sauer. Proc. Natl. Acad. Sci. USA. 98:2301-2305 {2001}). Even with this structural similarity, Arc and Mnt recognize almost completely different operator sequences with only 6 of 21 base pairs in common (Vershon et al. J. Mol. Biol. 195:323-31 {1987}; Vershon et al. J. Mol. Biol. 195:311-322 {1987}).

For the promoter/repressor system of the present invention, co-expression of two repressor proteins, the wildtype Mnt repressor and a mutant Mnt-Arc protein are utilized. The mutant Mnt-Arc proteins contains the wildtype C-terminal dimerization domain from Mnt; however, six residues within the N-terminal DNA binding domain have been replaced with the corresponding 9 residues from the Arc repressor (Knight and Sauer. Proc. Natl. Sci. USA 86:797-801 {1989}). A Mnt-Arc homodimer retains wildtype tetramerization ability, but now recognizes the Arc operator sequence (O₂) instead of the Mnt operator (01). The novel repressor heterotetramer of the present invention consists of one wildtype Mnt homodimer and one hybrid Mnt-Arc homodimer (pictured in FIG. 12).

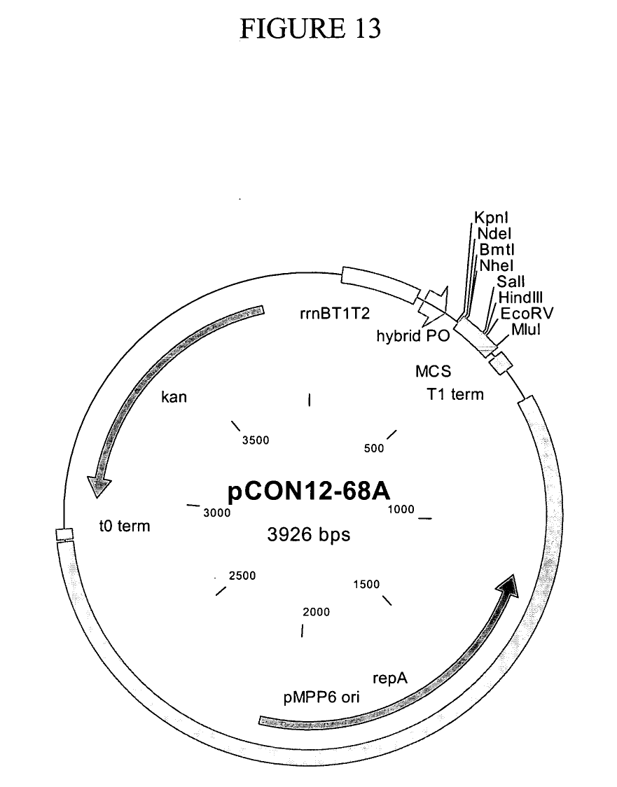

In some embodiments, the hybrid bacterial promoter consists of near-consensus σ⁷⁰ −35 and −10 hexamer sequences to achieve the highest level of transcription possible in the target bacteria. However, in other embodiments, alternate hexamer sequences are utilized to achieve optimal expression in non-E. coli bacterial hosts. In preferred embodiments, the rrnB T1T2 terminators, described above, are positioned upstream of the promoter, to provide protection against read-through transcription and the low copy modified-pSC101 replication origin (from pMPP6), which is maintained at 3-4 copies per cell (plasmid pCON12-68A) are utilized. FIG. 13 shows a map of one exemplary expression vector of the present invention that utilizes the hybrid promoter/operator described herein.

In preferred embodiments, the two operator half-sites 01 and O₂ for repressor protein binding are positioned so that they are downstream from the −35 and/or −10 hexamers; therefore, repressor binding will directly occlude RNA polymerase from initiating transcription. Experiments conducted during the course of development of the present invention demonstrated that the preferred positioning of 01 and O₂ operator half-sites utilizes directly adjacent operator sites. Because both operator half-sites are located downstream of the −35 and −10 hexamers, alternative “species-specific” promoters can be substituted without altering the repression ability of the Mnt and Mnt-Arc mutant repressors. The DNA sequence of the hybrid promoter is given in FIG. 14 (SEQ ID NO:13). When the operators 01 and O₂ are orientated properly on the DNA, the wildtype Mnt dimer and mutant Mnt-Arc dimer form a stable hetero-tetramer and bind the operators with high affinity and specificity. Stable binding of the hetero-tetramer to the “hybrid” operator strongly represses gene expression. Note that the wildtype Mnt or wildtype Arc repressors can not recognize the hybrid operator (O1-O2). They still can recognize each operator sequence (O1 or O2 independently), but due to lack of tetramer formation, these wildtype repressor proteins do not bind to the region tightly.

Acquisition of the Mnt and/or Arc repressors by pathogenic bacteria does not readily confer resistance to expression of toxic genes because of the following reasons: (1) The wild-type Mnt tetramer will not recognize the hybrid operator sequence. (2) The wild-type Arc tetramer will not recognize the hybrid operator sequence. (3) A Mnt-Arc protein formed by homologous recombination between acquired Arc and Mnt proteins will eliminate the wildtype copy, which is still required for repression. In addition, bacteriophage P22 is restricted to Salmonella species, and the chance of E. coli and other pathogens being exposed to the genes from this phage is less likely. The hybrid promoter/repressor system of the present invention is thus ideal for regulating the expression of genes and RNA in any bacterial species.

In additional preferred embodiments, the vectors of the present invention comprise a low copy number origin of replication (e.g., low copy modified pSC101 (SEQ ID NO:11) or RK2 (SEQ ID NO:12). The present invention is not limited to low copy modified pSC101 or RK2 origins of replication. Other exemplary origins of replication include, but are not limited to, wildtype pSC101, p15a, pACYC.

In additional embodiments, vectors comprise a multiple cloning site for insertion of nucleic acid encoding genes of interest and a selectable marker (e.g., an antibiotic resistance gene such as kanamycin, ampicillin, tetracycline, etc.). In still further embodiments, the vectors of the present invention comprise protein purification tags (e.g., His-tag, intein tag). In some embodiments, the ribosome binding site is modified to allow increased/decreased translation.

II. The Present Invention in Operation

The vectors of the present invention constitute a tightly regulated expression system for the cloning and expression of genes in E. coli and closely related bacteria.

A. Expression

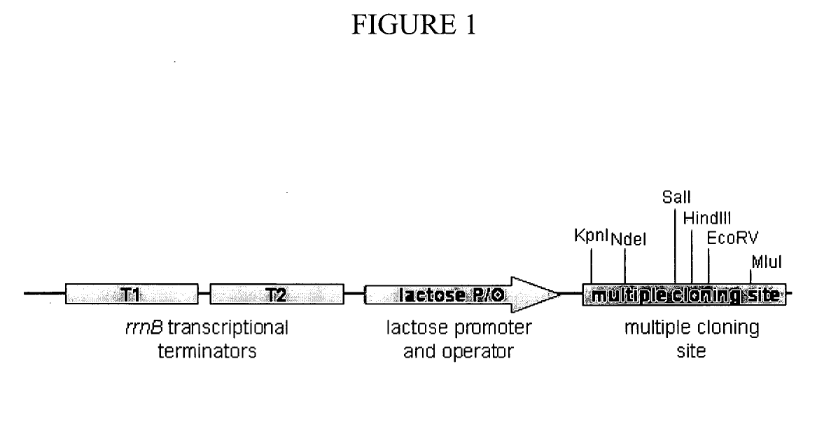

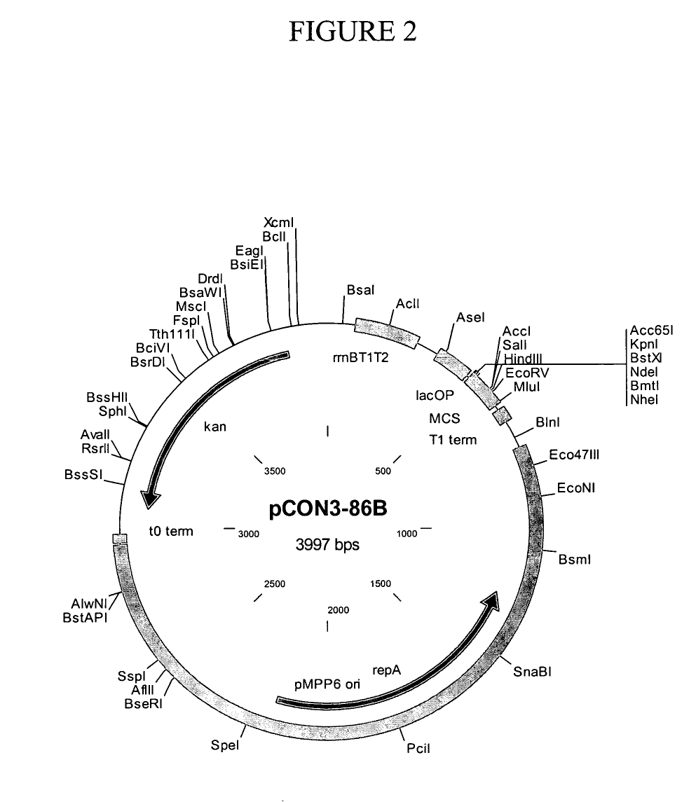

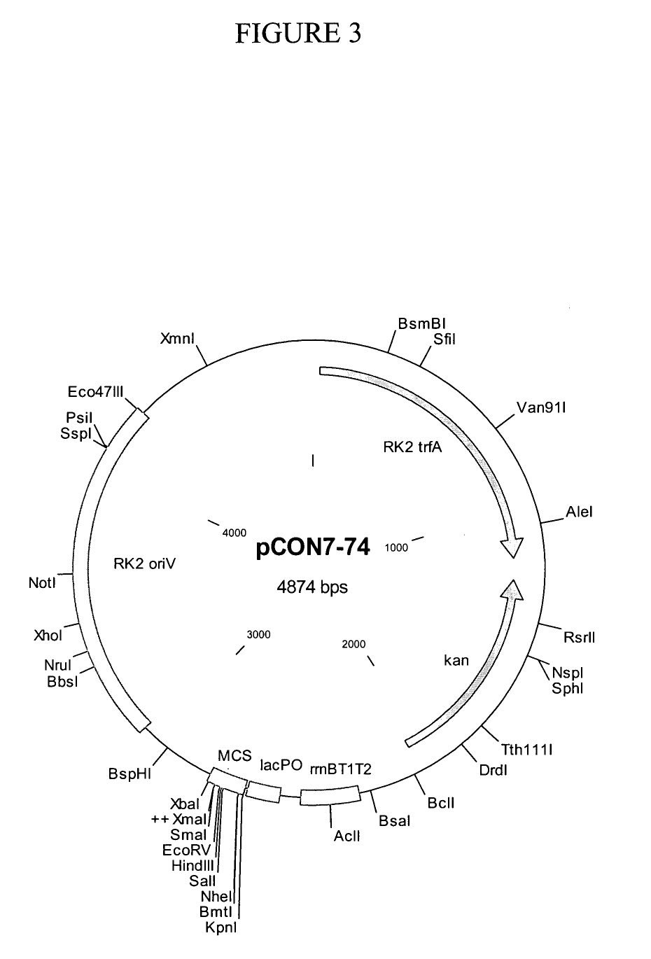

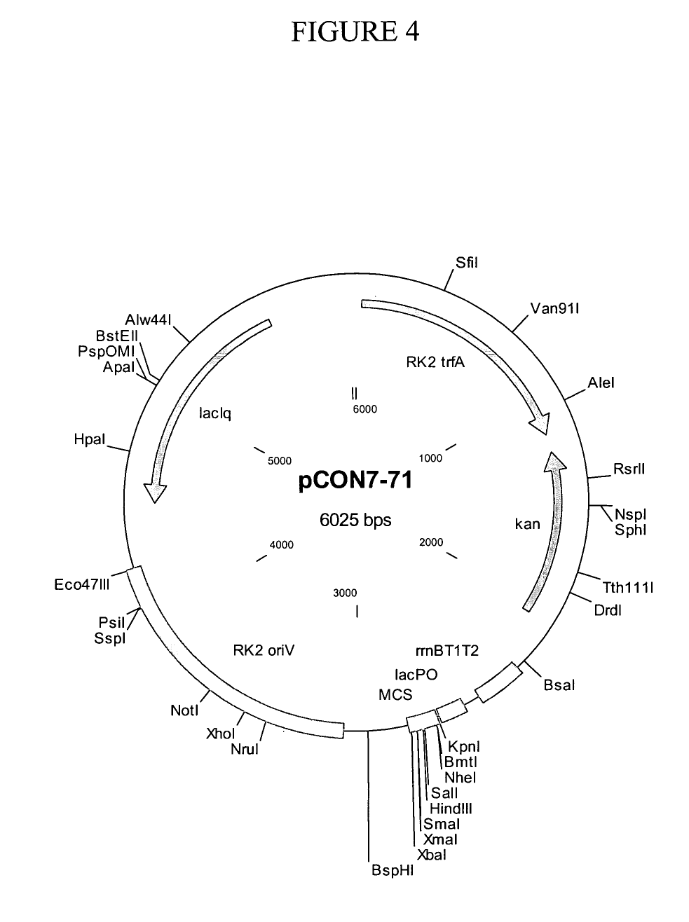

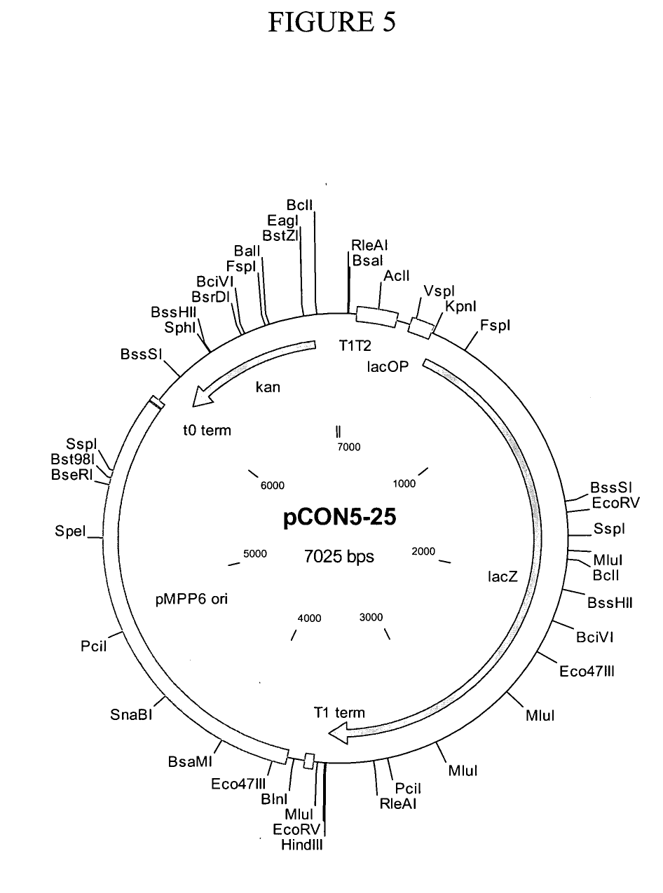

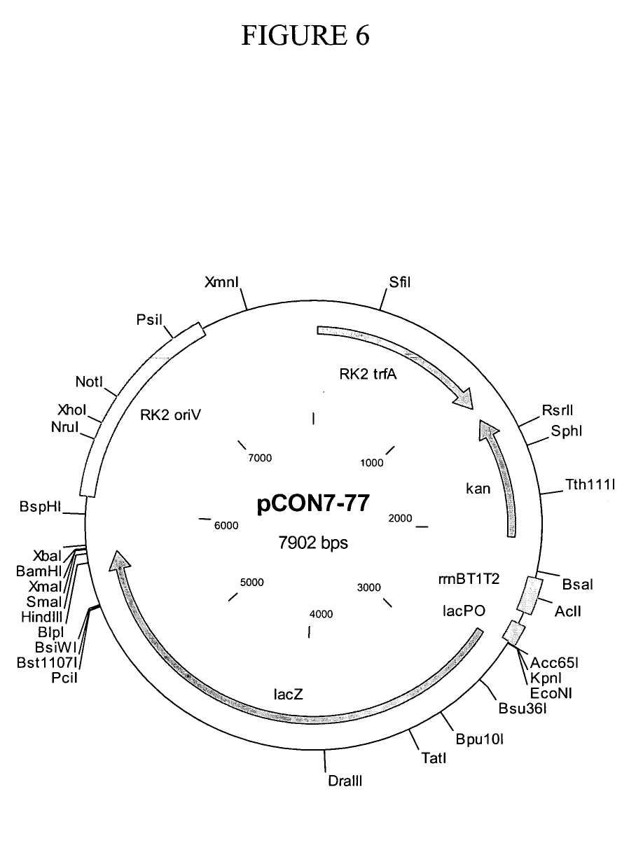

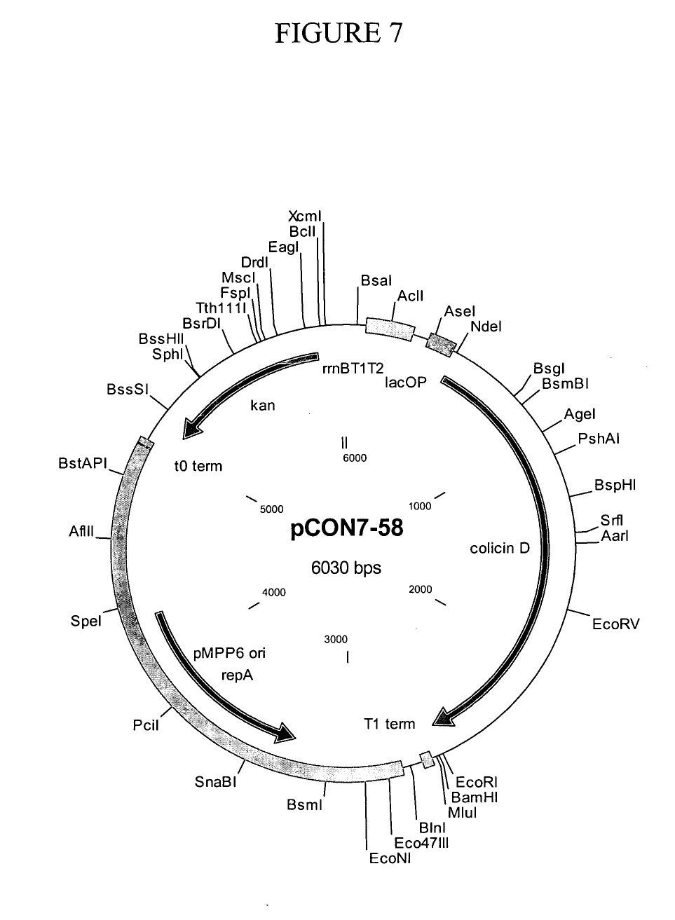

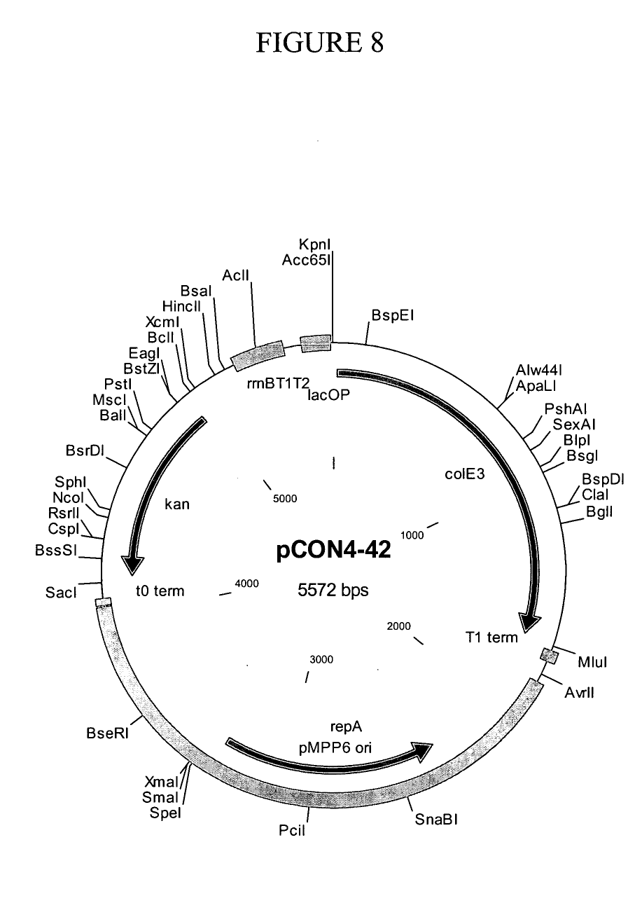

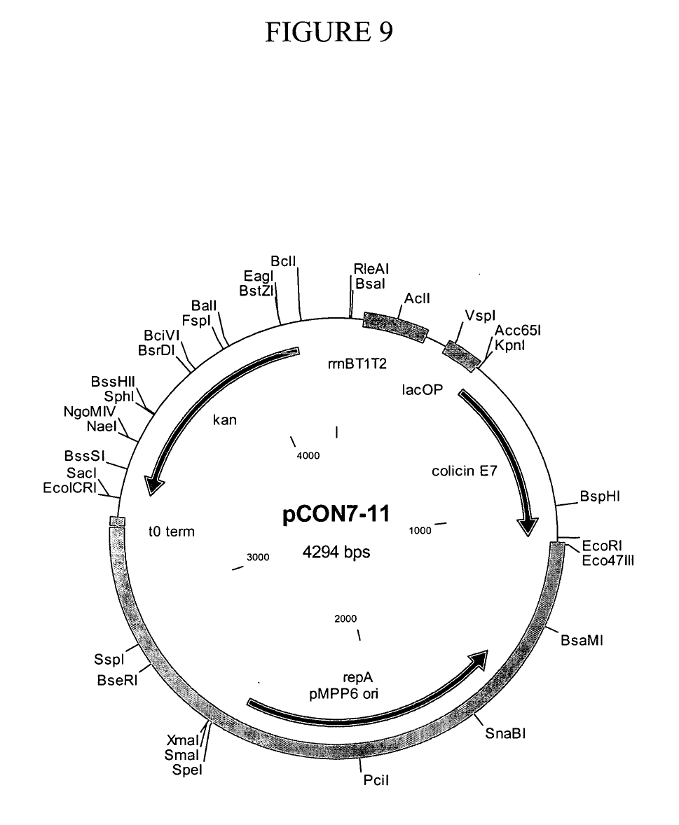

FIGS. 1 and 13 describe exemplary vectors of the present invention. The gene of interest is cloned into the multiple cloning site (MCS in FIG. 1) under control of the wildtype lactose promoter (lacOP in FIG. 1). This promoter is repressed by the lactose repressor protein (LacI) which is supplied either on the chromosome, an F′ element, and/or on a second plasmid. Upon induction with IPTG or removal of the LacI repressor protein, the lactose promoter becomes de-repressed and leads to strong expression of the cloned gene. In other embodiments, the hybrid mutant Mnt-Arc promoter operator system is utilized. The promoter is protected from read-through transcription and “leaky” expression by the ribosomal rrnB T1 and T2 transcriptional terminators (rrnBT1T2 in FIG. 1). When positioned upstream of the promoter region, these terminators are extremely efficient at preventing transcriptional read-through into the promoter region. In some embodiments, the expression system utilizes the low copy modified-pSC101 replication origin (from pMPP6), which is maintained at 3-4 copies per cell. This low copy number further minimizes any “leaky” expression of the cloned gene. In other embodiments, the origin of replication from the low copy RK2 replication origin, which can replicate in a wide variety of Gram negative bacteria is utilized. The RK2 replication origin allows this expression system to be used not only in E. coli, but in bacteria ranging from pathogens to bacteria used in industrial applications. The low copy number of RK2 further minimizes any “leaky” expression of the cloned gene.

The vectors of the present invention are suitable for the expression of any protein or RNA in a bacterial host. However, the combination of low copy number and tightly controlled expression make the plasmids particularly suitable for the maintenance, replication and expression of toxic proteins, toxic RNAs, and proteins with toxic metabolites. The vectors of the present invention also permit the expression of toxic proteins that might otherwise result in cell death from leaky expression. Experiments conducted during the course of development of the present invention (see, e.g., Example 3) demonstrated the cloning, maintenance, and expression of toxin colicin proteins.

The vectors of the present invention are suitable for use with a variety of toxic proteins, RNAs, and proteins with toxic metabolites. For example, in some embodiments, the vectors of the present invention find use in the expression of anti-microbial agents (e.g., antibiotics). Agents may include protein or peptide agents such as cationic-rich antibacterial peptides, proline-rich antibacterial peptides, colicins, bacteriocins, defensins, ricin, pyrrhocoricin, pexiganan, lsegagan, protegrin-1, thanatin, astacidin 1, sarcotoxin IA, and microcin J25. Agents may also include RNA-based compounds such as antisense RNA, microRNAs (mRNAs), small interfering RNAs (siRNAs), catalytic RNAs, and RNA aptamers.

In a further embodiment, the present invention provides bacterial host cells containing the above-described constructs. Specific examples of host cells include, but are not limited to, Escherichia coli, Salmonella typhimurium, Bacillus subtilis, and various species within the genera Helicobacter, Pseudomonas, Streptomyces, and Staphylococcus.

The constructs in host cells can be used in a conventional manner to produce the gene product encoded by the recombinant sequence. In some embodiments, introduction of the construct into the host cell can be accomplished by calcium phosphate transfection, DEAE-Dextran mediated transfection, or electroporation (See e.g., Davis et al., Basic Methods in Molecular Biology, {1986}).

In some embodiments of the present invention, following transformation of a suitable host strain and growth of the host strain to an appropriate cell density, the selected promoter is induced by appropriate means (e.g., temperature shift or chemical induction) and cells are cultured for an additional period. In other embodiments of the present invention, cells are typically harvested by centrifugation, disrupted by physical or chemical means, and the resulting crude extract retained for further purification. In still other embodiments of the present invention, microbial cells employed in expression of proteins can be disrupted by any convenient method, including freeze-thaw cycling, sonication, mechanical disruption, or use of cell lysing agents.

The present invention also provides methods for recovering and purifying proteins expressed from recombinant cell cultures comprising a vector of the present invention including, but not limited to, ammonium sulfate or ethanol precipitation, acid extraction, anion or cation exchange chromatography, metal ion chelate chromatography, phosphocellulose chromatography, hydrophobic interaction chromatography, affinity chromatography, hydroxylapatite chromatography and lectin chromatography. In some preferred embodiments, methods for recovering and purifying said proteins comprise metal ion chelate chromatography or affinity chromatography selected to interact with a purification tag (e.g., His tag or intein tag) on the protein. In other embodiments of the present invention, protein-refolding steps can be used as necessary, in completing configuration of the mature protein. In still other embodiments of the present invention, high performance liquid chromatography (HPLC) can be employed for final purification steps.

B. Kits

In some embodiments, the present invention provides kits comprising a vector of the present invention. As used herein, the term “kit” refers to any delivery system for delivering materials. In the context of cloning and expression systems, such delivery systems include systems that allow for the storage, transport, or delivery of cloning components and/or supporting materials (e.g., buffers, written instructions for using the components, etc.) from one location to another. In some embodiments, the kits comprise all of the components necessary to clone a gene (e.g., a gene encoding a toxic protein), for example, including, but not limited to, vector, buffers, salts, enzymes, controls and instruction for using the kit for cloning. In some additional embodiments, the kit further comprises components for cloning and expressing a gene of interest. Additional components useful for gene expression include control plasmids for quantitating gene expression levels, as well as components for protein purification (e.g., resins and buffers).

### EXPERIMENTAL

The following examples are provided in order to demonstrate and further illustrate certain preferred embodiments and aspects of the present invention and are not to be construed as limiting the scope thereof.

### Example 1

### Plasmid Construction

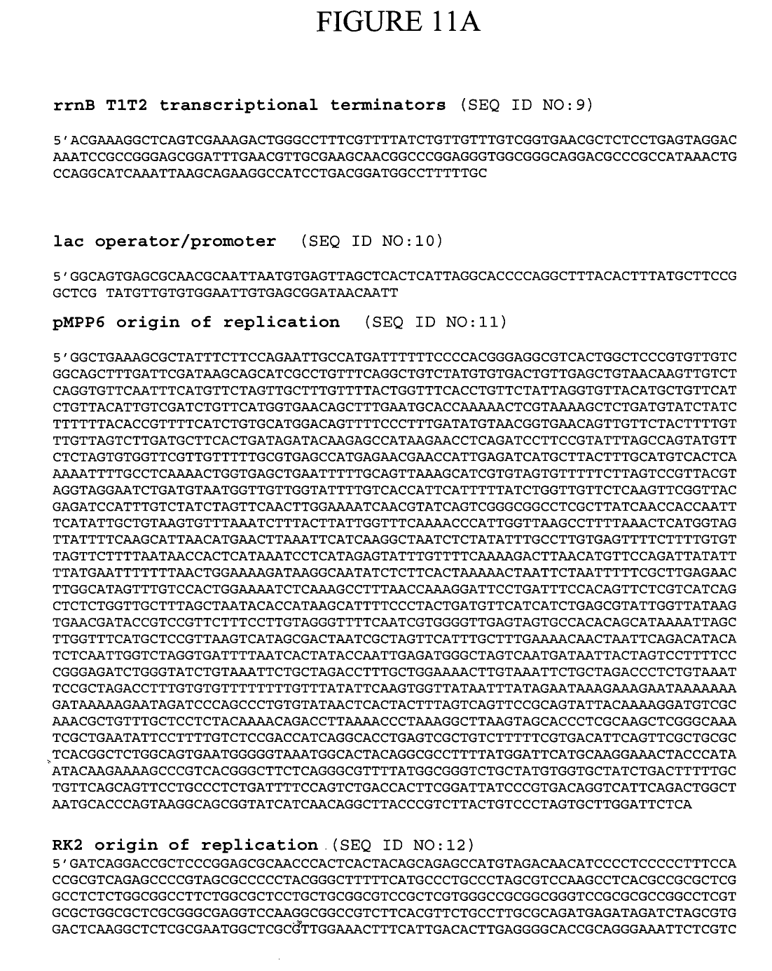

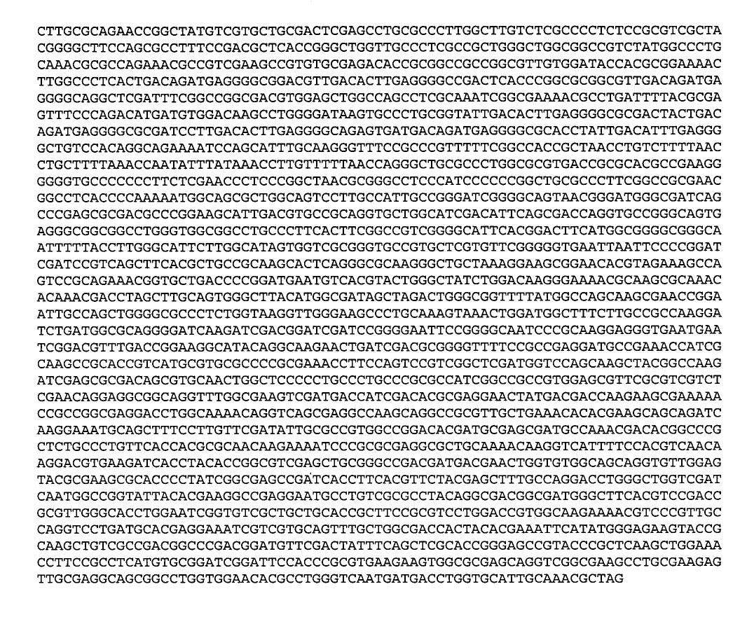

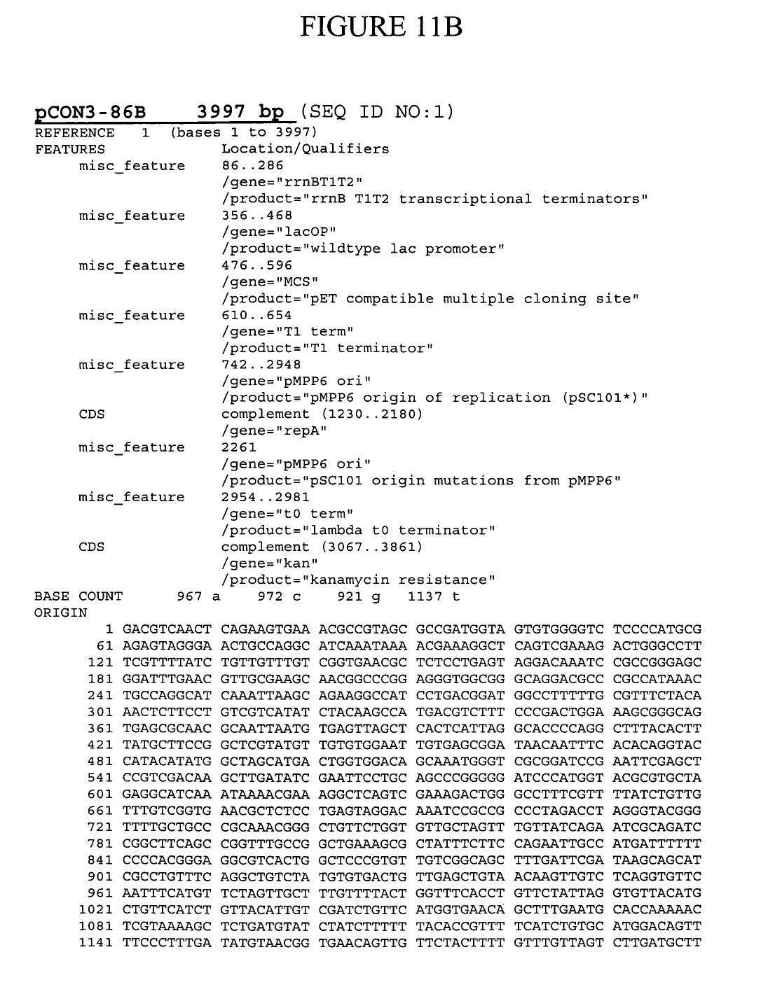

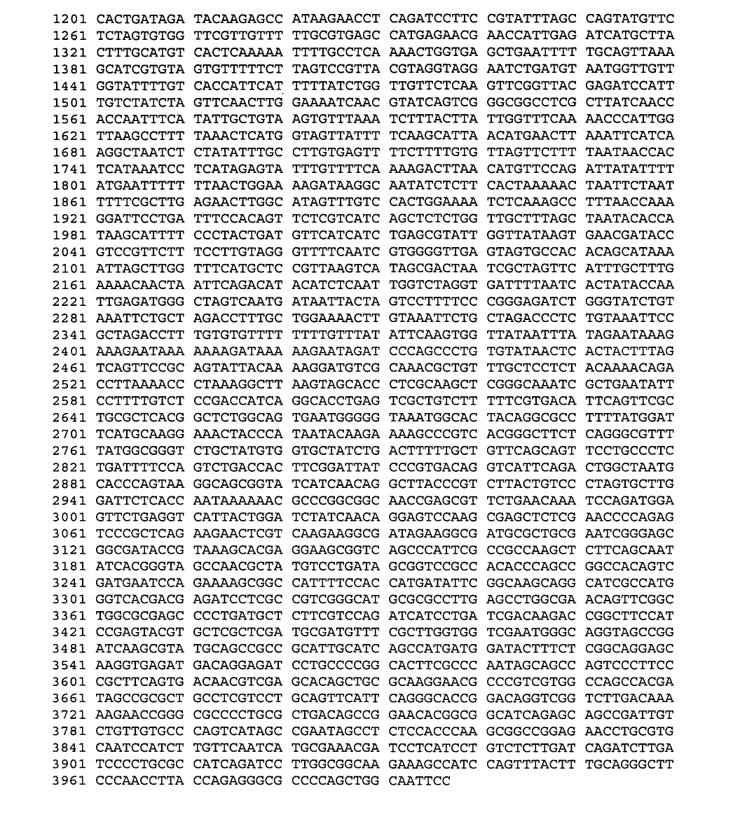

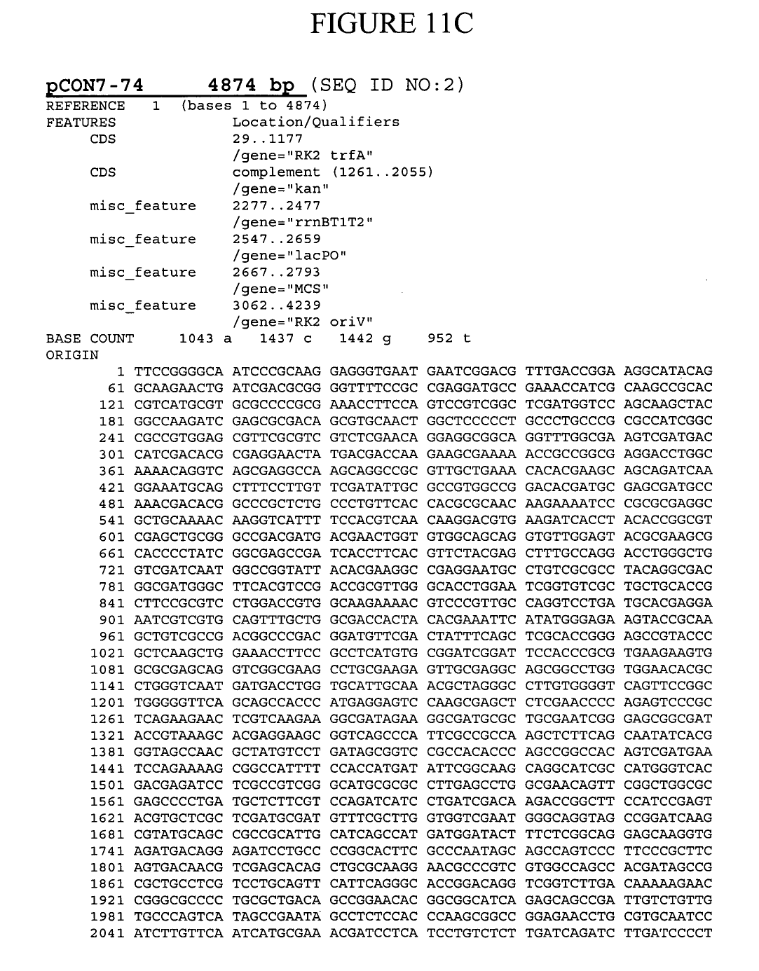

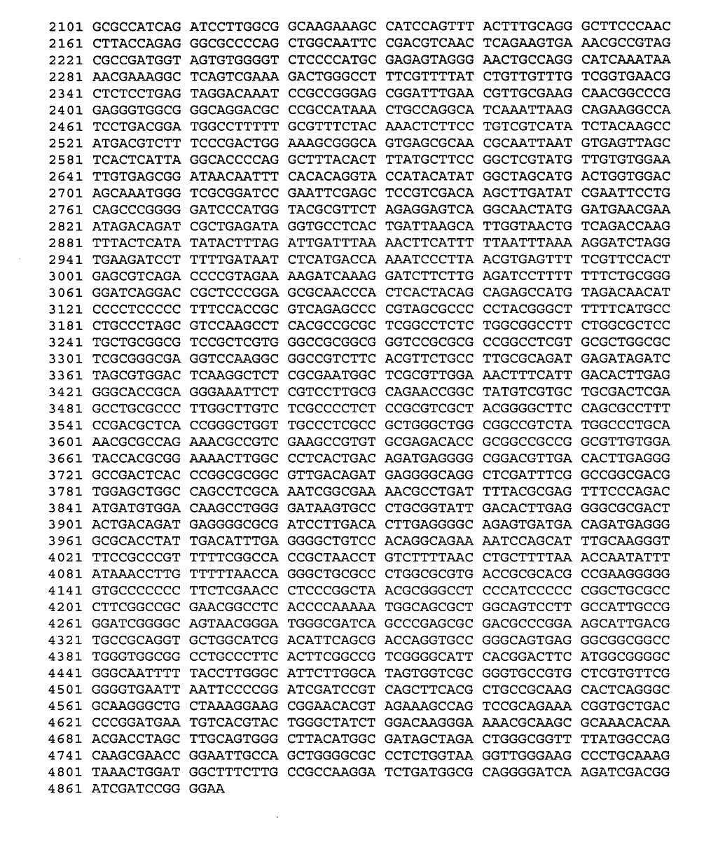

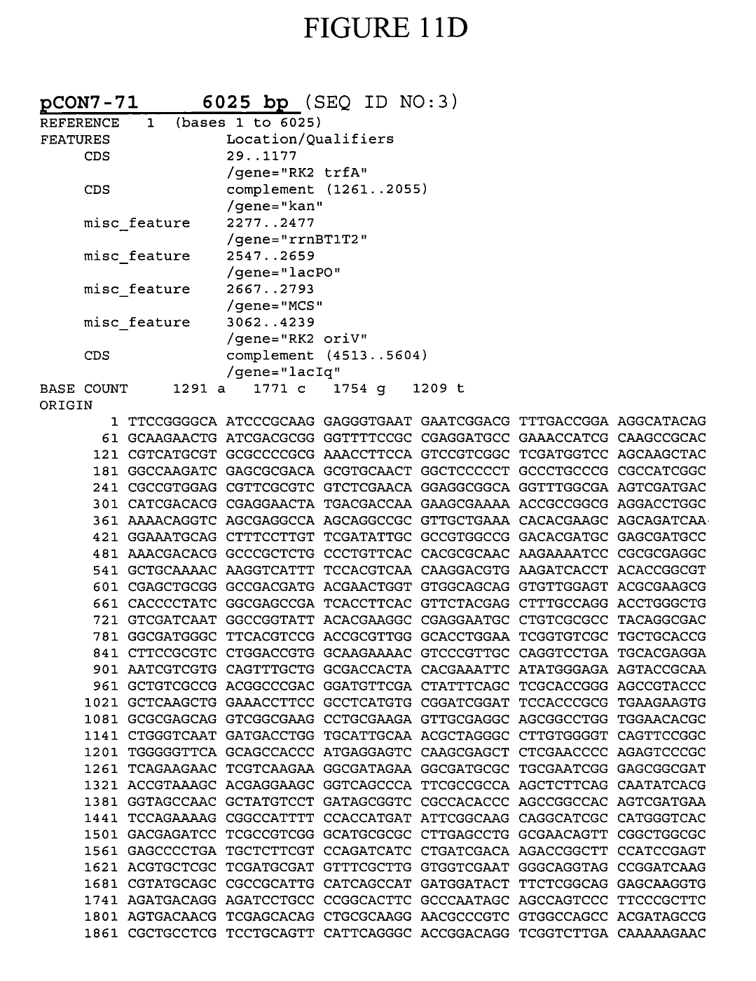

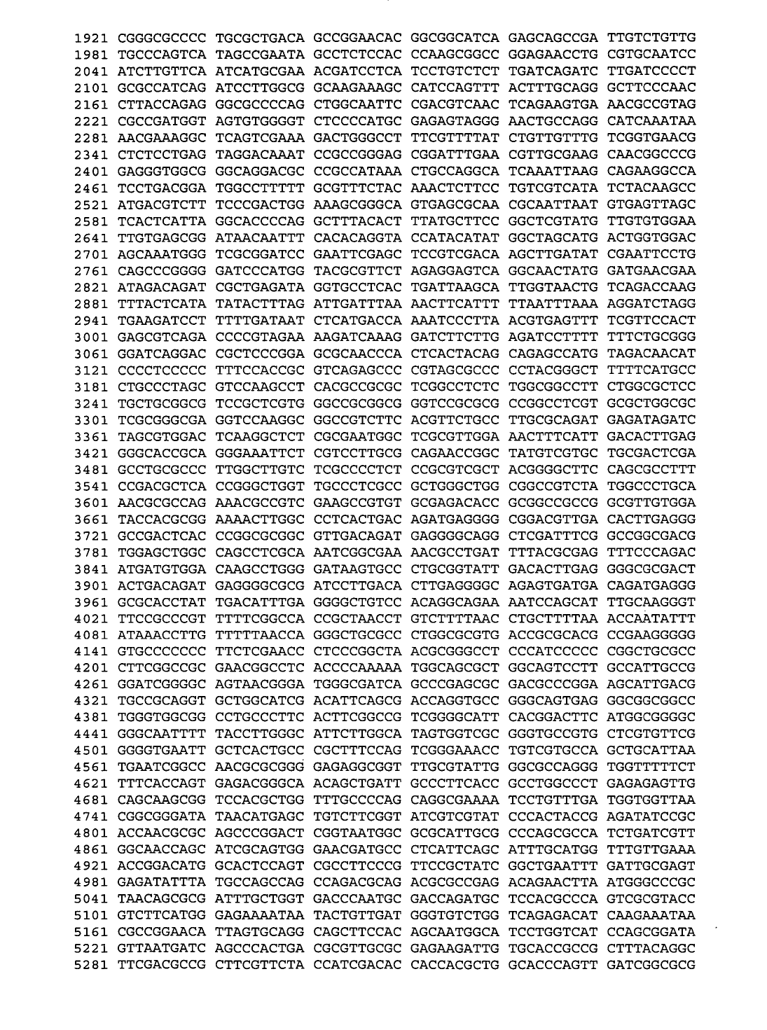

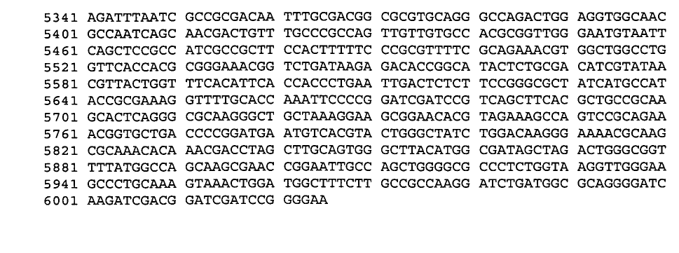

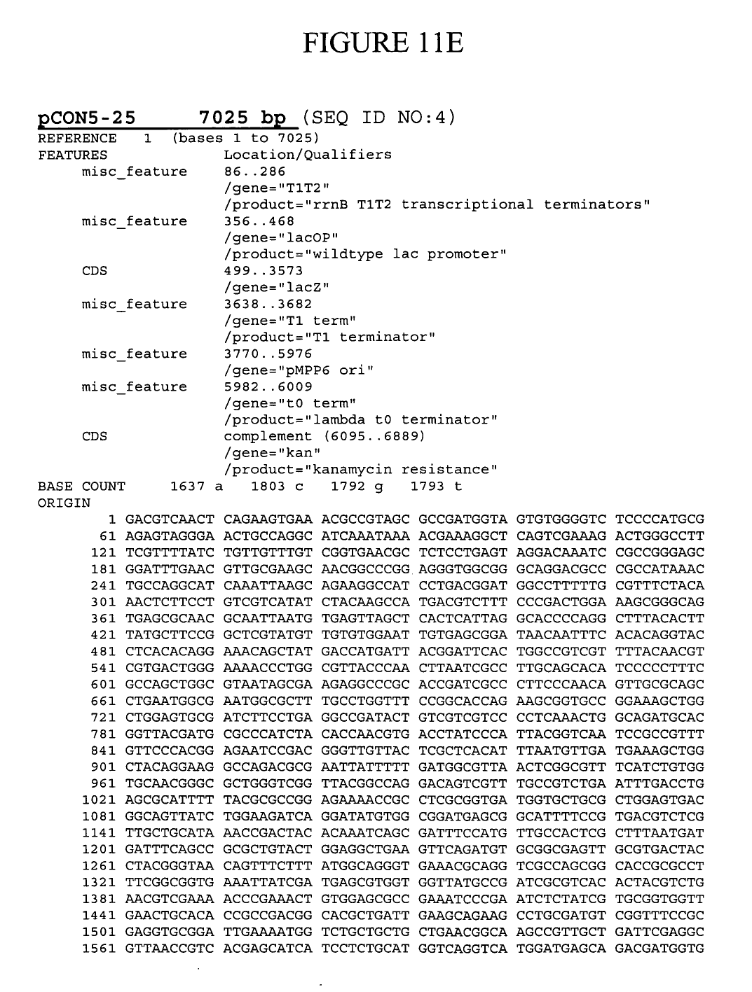

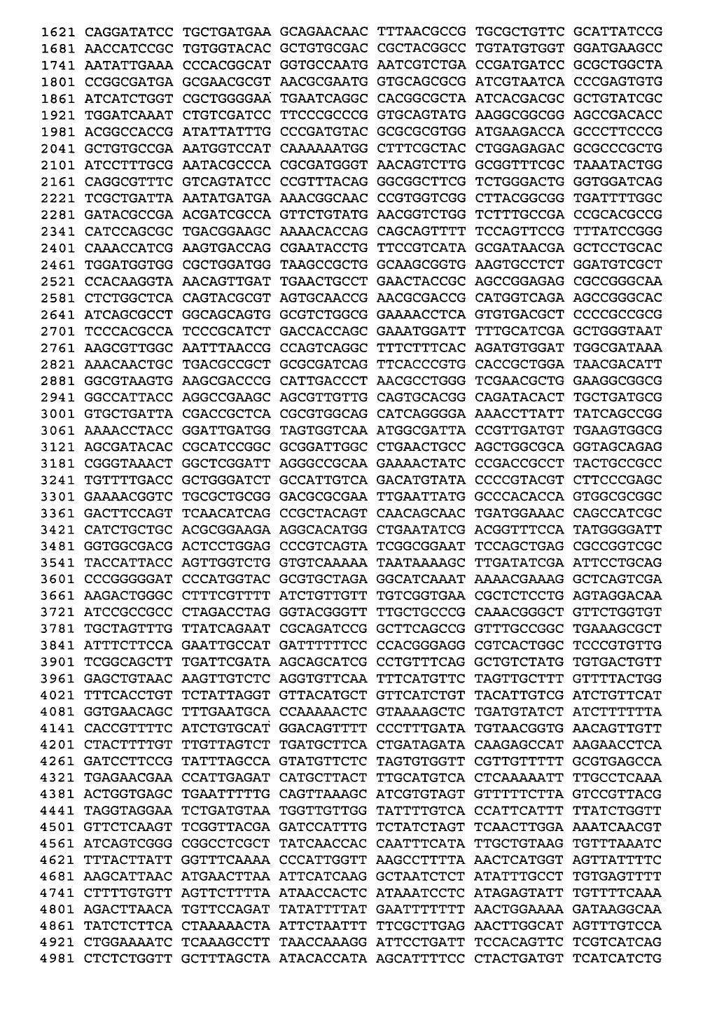

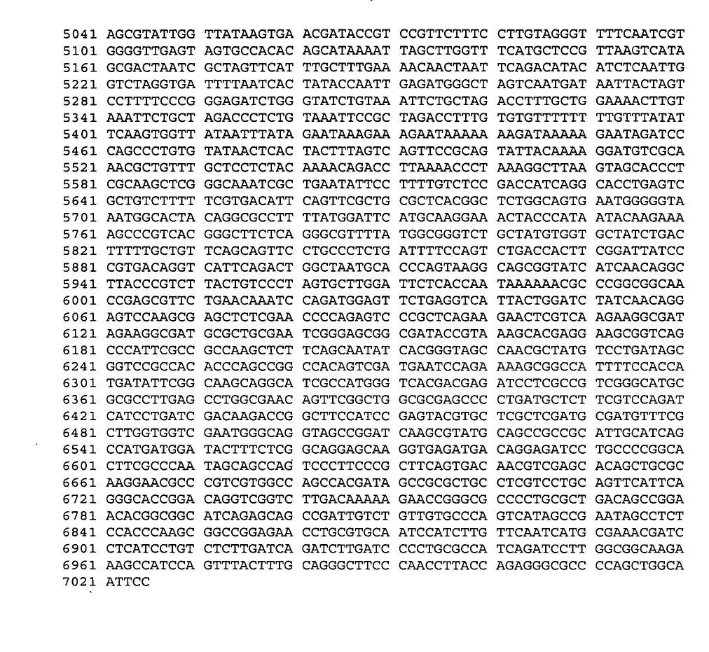

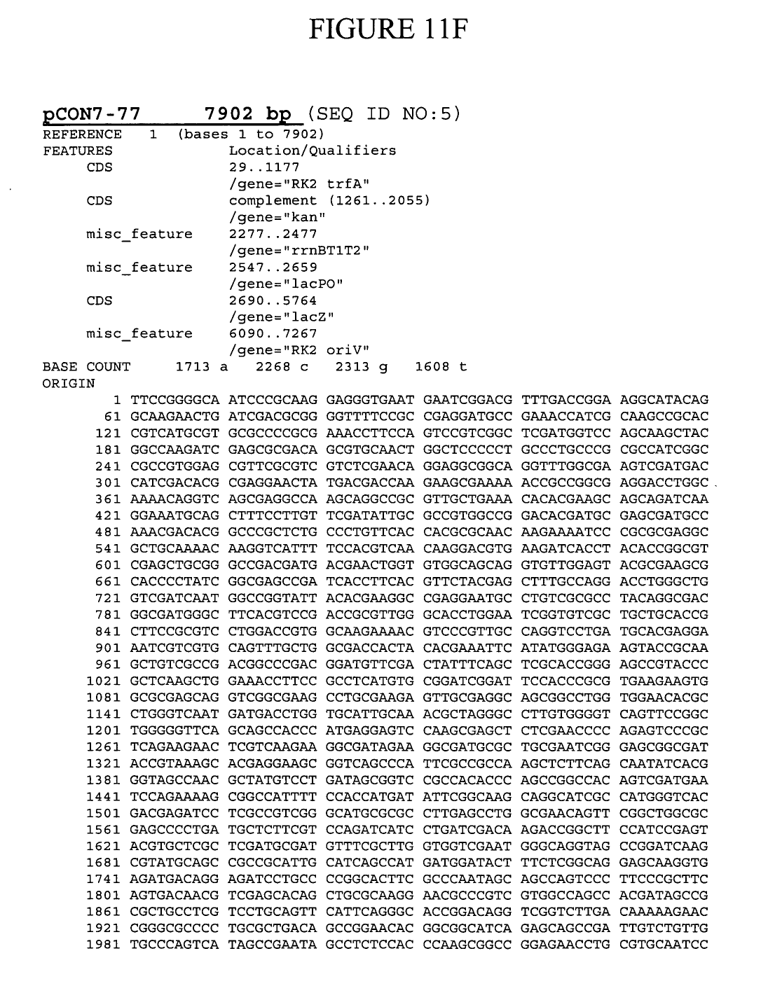

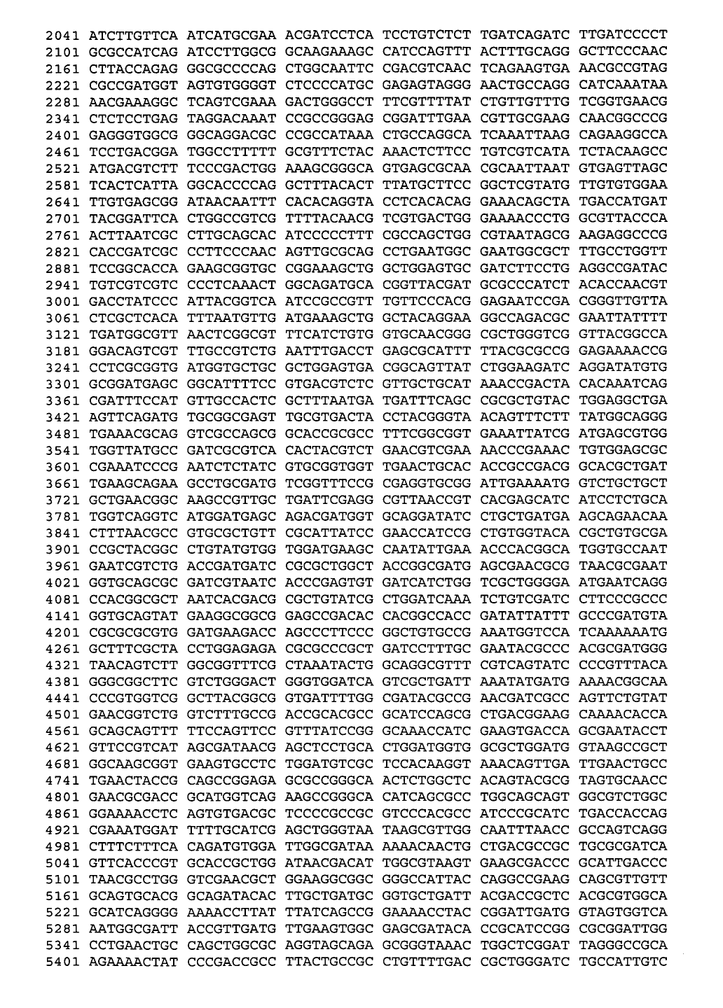

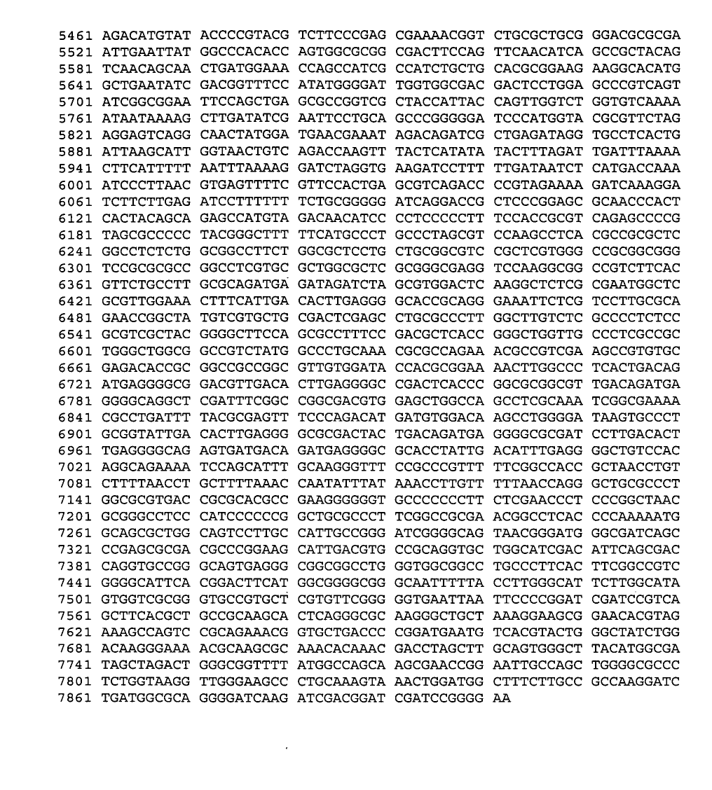

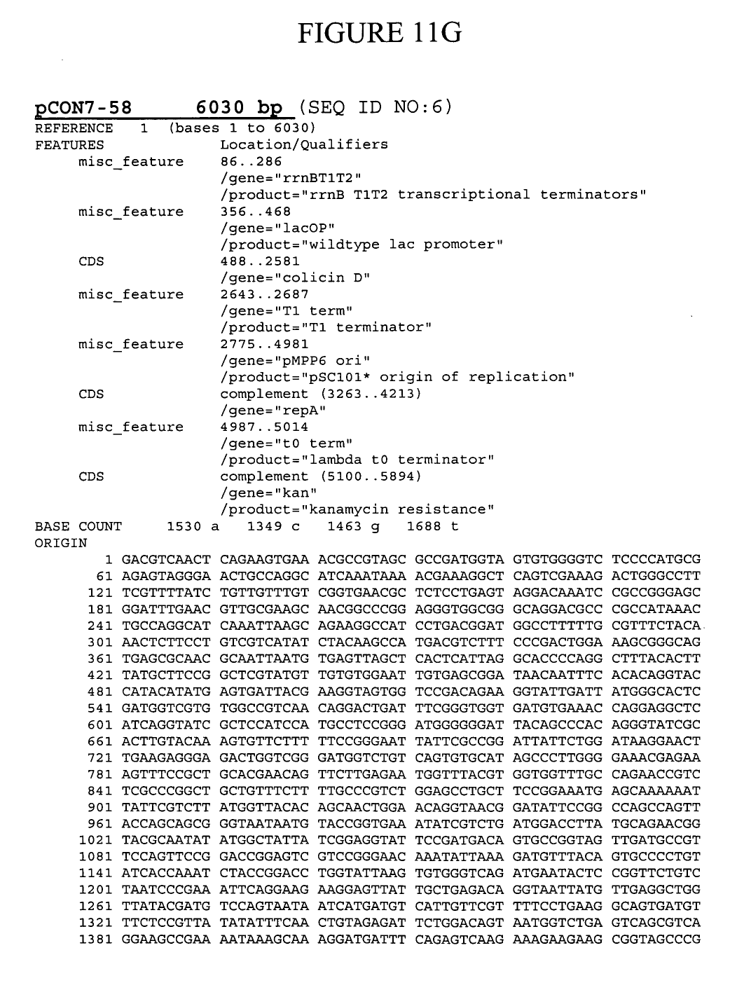

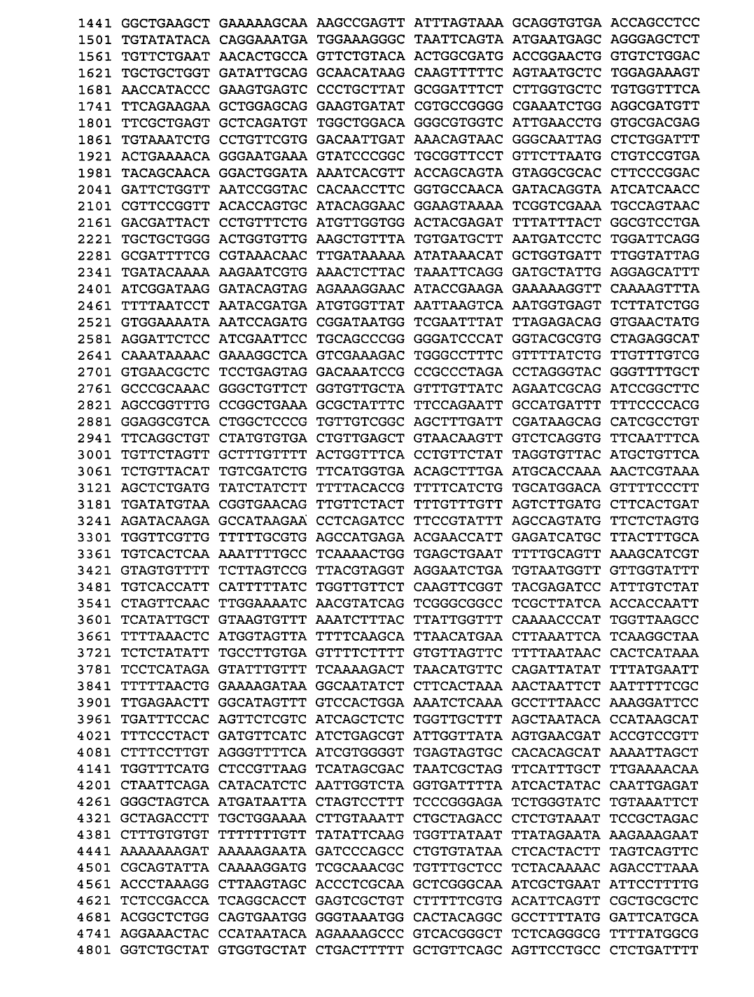

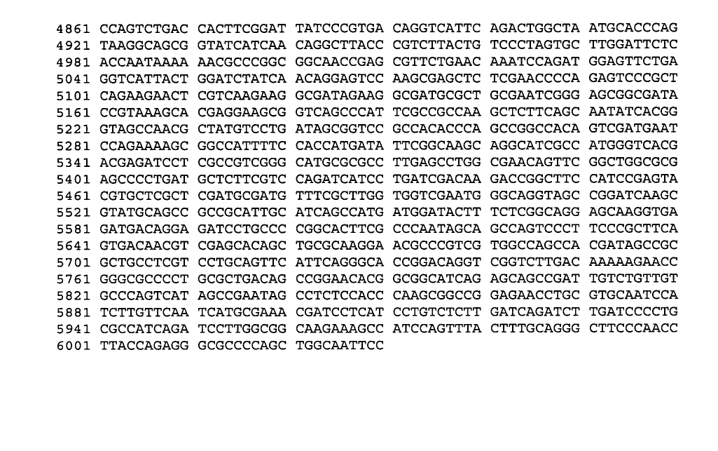

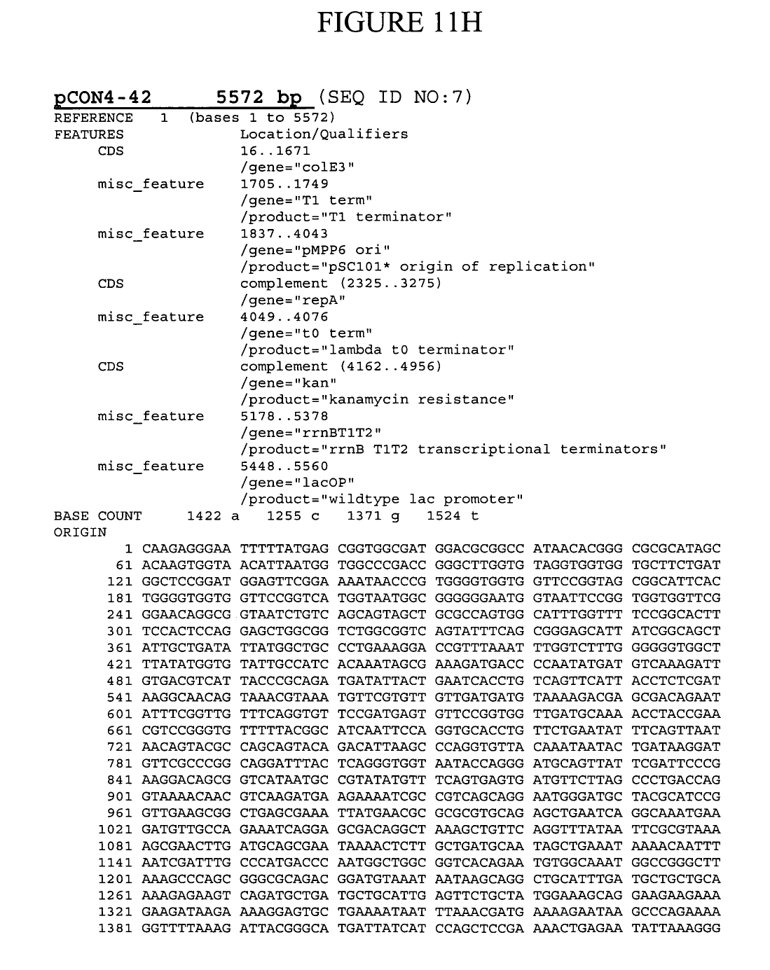

This Example describes the construction of exemplary plasmids of the present invention. Table 1 shows the names and corresponding Figure and SEQ ID NO designations for the plasmids described below. Sequences of plasmids and selected vector elements are shown in FIG. 11.

TABLE 1
Plasmids

| Name | Figure (depicting map) | SEQ ID NO |
|---|---|---|
| pCON3-86B | 2 | 1 |
| pCON7-74 | 3 | 2 |
| pCON7-71 | 4 | 3 |
| pCON5-25 | 5 | 4 |
| pCON7-77 | 6 | 5 |
| pCON7-58 | 7 | 6 |
| pCON4-42 | 8 | 7 |
| pCON7-11 | 9 | 8 |

A. Materials and Methods
Bacterial Strains and Media

The Escherichia coli strain utilized was NovaBlue {endA1 hsdR17(rK12-mK12+) supE44 thi-1 recA1 gyrA96 relA1 Δlac F′(proA+B+lacIqZAM15::Tn10 (Tc^{R}))} from Novagen (Madison, Wis.). All cloning was performed using standard methods known in the art, and using Luria Bertani growth media supplemented with 50 μg/ml kanamycin to permit selection for plasmids. For cloning of toxic gene products such as the colicins, the growth media was supplemented with 0.8% glucose to further repress the lactose promoter.

B. Plasmid Construction

Construction of pCON3-86B

The DNA region that contains the pMPP6 origin of replication and kanamycin resistance gene was derived from plasmid pZS24-MCS1 (Lutz and Bujard, Nucleic Acids Res. 25(6):1203-1210 {1997}; Manenet al., Mol Microbiol 11(5):875-884 {1994}). The internal Nde I restriction site in the pMPP6 origin was removed by site-directed mutagenesis. The wildtype lactose promoter was PCR amplified from E. coli K12 MG1655 genomic DNA and combined with the pMPP6 origin and kanamycin resistance gene via Aat II and Kpn I restriction sites. The rrnB ribosomal terminators T1 and T2 were PCR amplified from plasmid pRLG593 (Ross et al., J Bacteriol 180:5375-83 {1998}; Glaser et al., 302:74-6 {1983}) and subcloned into the vector, resulting in plasmid pCON3-86B.

Construction of pCON7-74

The DNA region of pCON3-86B that contains the kanamycin resistance gene, rrnB terminators, lactose promoter, and multiple cloning site was PCR amplified and subcloned into the mini-RK2 vector pCON4-43 via Nco I and Mlu I restriction sites. The resulting construct is pCON7-74.

Construction of pCON7-71

The DNA region encoding lacIq gene was PCR amplified from plasmid pCON1-94 and subcloned into pCON7-74 via the Xmn I restriction site. The resulting construct is pCON7-71.

Construction of pCON5-25

The DNA region encoding lacZ was PCR amplified from E. coli K12 MG1655 genomic DNA and subcloned into pCON3-86B via Kpn I and Hind III restriction sites. The resulting vector is pCON5-25.

Construction of pCON7-77

The DNA region encoding lacZ was PCR amplified from E. coli K12 MG1655 genomic DNA and subcloned into pCON7-74 via Kpn I and Hind III restriction sites. The resulting vector is pCON7-77.

Construction of pCON7-58

The DNA region encoding colicin D was PCR amplified from the plasmid pColD-CA23 (Lehrbach and Broda, JGen Microbiol 130:401-10 {1984}) and subcloned into pCON3-86B via Nde I EcoRV restriction sites. Transformants were plated on LB media supplemented with 50 μg/ml kanamycin and 0.8% glucose. The resulting vector is pCON7-58.

Construction of pCON4-42

The DNA region encoding colicin E3 was PCR amplified from the plasmid pColE3-CA38 (Vemet et al., Gene 34(1):87-93 {1985}) and subcloned into pCON3-86B via Kpn I Mlu I restriction sites. Transformants were plated on LB media supplemented with 50 μg/ml kanamycin and 0.8% glucose. The resulting vector is pCON4-42.

Construction of pCON7-11

The DNA region encoding colicin E7 was PCR amplified from the plasmid pColE7-K317 (Watson et al., J Bacteriol 147(2):569-77 {1981}) and subcloned into pCON3-86B via Kpn I EcoRI restriction sites. Transformants were plated on LB media supplemented with 50 μg/ml kanamycin and 0.8% glucose. The resulting vector is pCON7-11.

### Example 2

### Gene Expression

This example describes the measurement of levels of expression from the vectors described in Example 1.

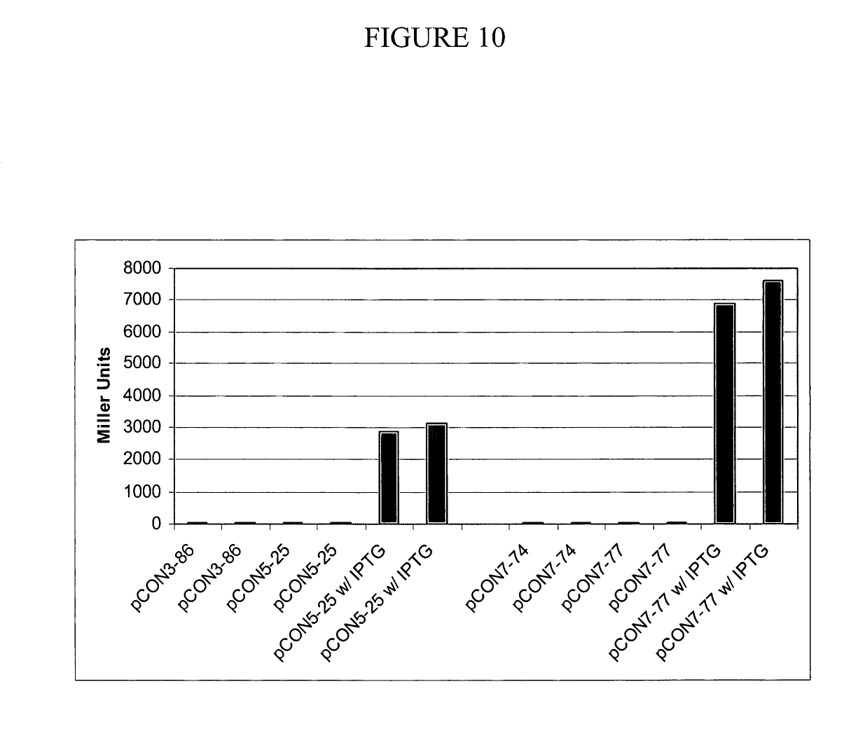

Using the standard assay for β-galactosidase activity, the promoter activity for vectors pCON3-86B, pCON5-25, pCON7-74, and pCON7-77 were obtained in repression conditions (Luria-Bertani broth supplemented with 0.8% glucose and 50 μg/ml kanamycin) and expression conditions (Luria-Bertani broth supplemented with 1 mM IPTG and 50 μg/ml kanamycin). Cultures were assayed in duplicate at an OD600 nm of 0.3-0.5 and expressed as Miller Units. The results are shown in FIG. 10.

As observed in FIG. 10, the promoter activities of pCON5-25 and pCON7-77 in repression medium are not significantly different from vectors pCON3-86B and pCON7-74, which do not contain the gene for α-galactosidase. However, upon de-repression with 1 mM IPTG, the promoter activity of pCON5-25 (with modified-pSC101 origin) is increased approximately 50-fold and the activity of pCON7-77 (with RK2 origin) is increased approximately 140-fold. These experiments demonstrate the tightness of control associated with these vectors.

### Example 3

### Expression of Toxic Proteins

The vectors of the present invention were used to clone and stably maintain the genes encoding colicins D (pCON7-58), E3 (pCON4-42), E7 (pCON7-11), E3 (pCON12-82) in the absence of the cognate immunity proteins, with the ability to achieve high levels of protein/RNA expression upon de-repression of the promoter.

### Example 4

### Construction of Vectors Containing the Wildtype Mnt and Mutant Mnt-Arc Repressor

This Example describes the construction of expression vectors comprising wildtype Mnt and mutant Mnt-Arc repressor. FIG. 12 shows a schematic of the hybrid promoter/operator of the present invention. FIG. 14 shows the nucleic acid sequence of the hybrid promoter (SEQ ID NO:13).

The mnt gene, encoding for wildtype Mnt repressor, was PCR-amplified from P22 phage DNA and subcloned into pCON7-42. In the resulting construct pCON9-53, the mnt gene is constitutively expressed from a strong promoter positioned upstream in the vector backbone.

A vector containing the mutant Mnt-Arc repressor was created as follows. A SphI site was introduced into pCON9-53 by site-directed mutagenesis, creating plasmid pCON12-35. The N-terminal residues of Mnt were removed by digesting pCON12-35 with KpnI SphI. An oligonucleotide linker cassette, containing the N-terminal 9 residues of Arc repressor, was subcloned into the digested pCON12-35 backbone by KpnI SphI digest. The resulting vector, which constitutively expresses mnt-arc, is pCON12-44.

Plasmid pCON12-55, which contains both mnt and mnt-arc genes, was created as follows. The promoter-mnt-arc cassette was PCR-amplified from pCON12-44 with flanking SpeI SacI restriction sites. This digested fragment was then subcloned directly into pCON9-53, resulting in plasmid pCON12-55.

Construction of “Hybrid” Promoter/Operator:

An oligonucleotide containing the “hybrid” promoter/operator with flanking AatII KpnI sites was used as a template for Klenow synthesis of the complementary strand. The dsDNA fragment was digested with AatII KpnI, and subcloned into the pMPP6 ori backbone (modified pSC101 origin). The resulting plasmid was pCON12-25E. The rrnB T1T2 terminators were removed from pCON3-86B by AatII KpnI digest, and subcloned into pCON12-25E, creating the expression vector pCON12-68A (shown in FIG. 13). pCON12-68A contains: rrnBT1T2 transcriptional terminators, “hybrid” promoter/operator, multiple cloning site, modified pSC101 origin of replication, and kanamycin resistance gene.

Cloning of lacZ and coIE3 Genes:

The lacZ gene encoding beta-galactosidase was removed from pCON5-25 by digestion with KpnI HindIII and subcloned into pCON12-25E, resulting in plasmid pCON12-29E.

The colE3 gene encoding Colicin E3 was removed from pCON4-42 by KpnI EcoRI and subcloned into pCON12-68A, resulting in plasmid pCON12-82.

Results

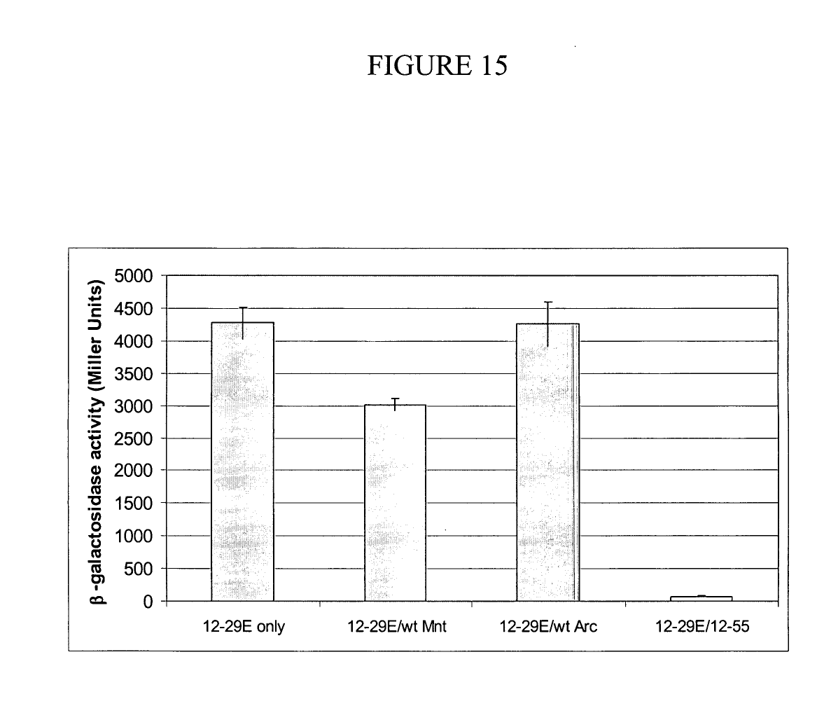

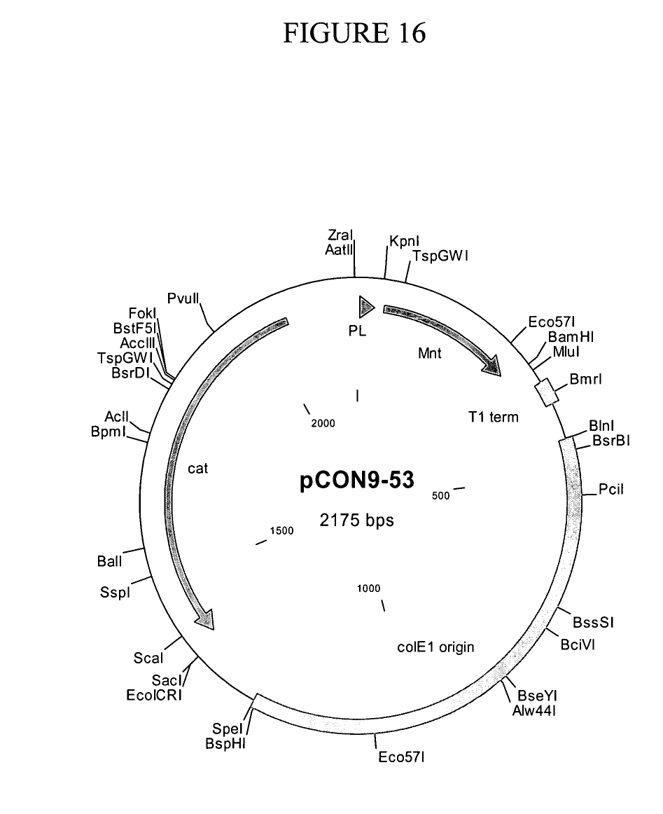

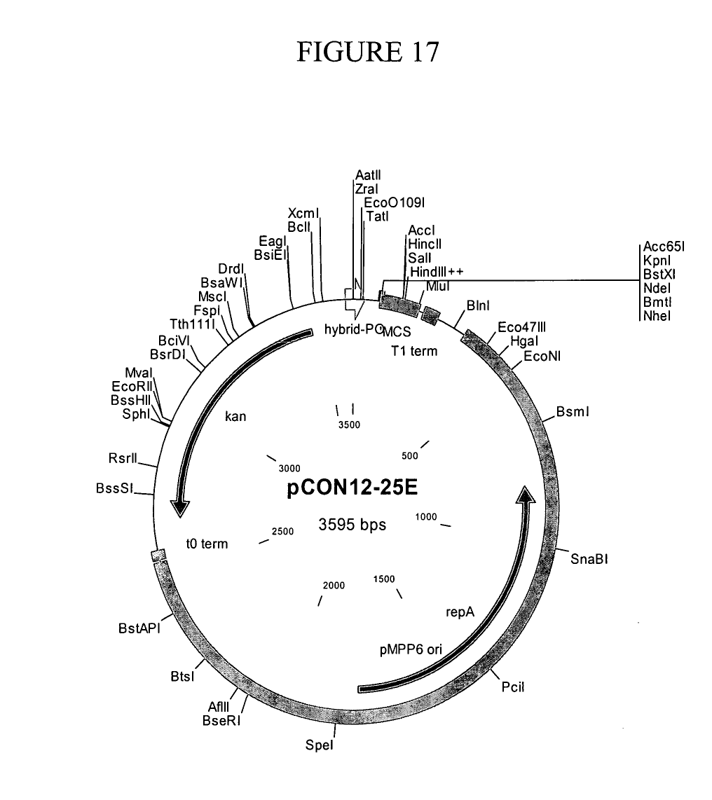

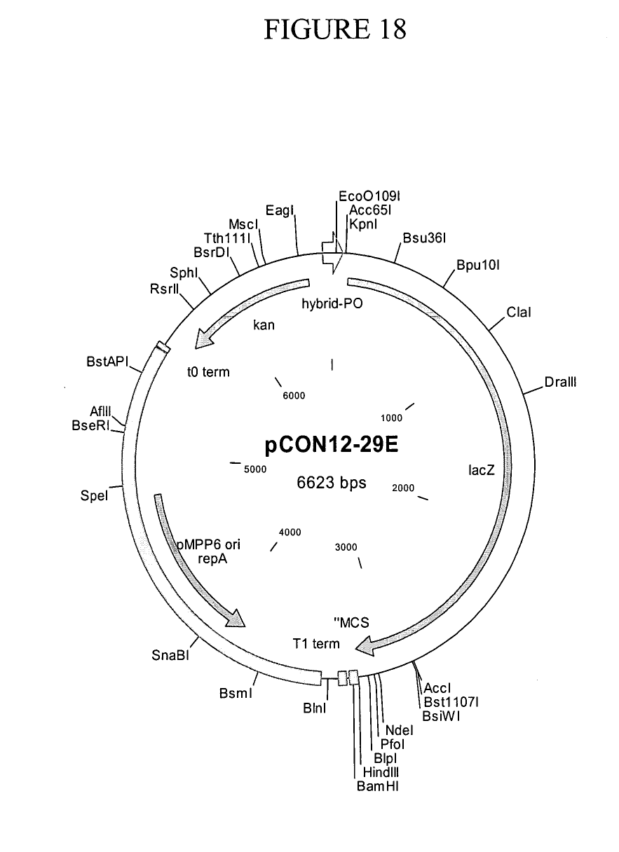

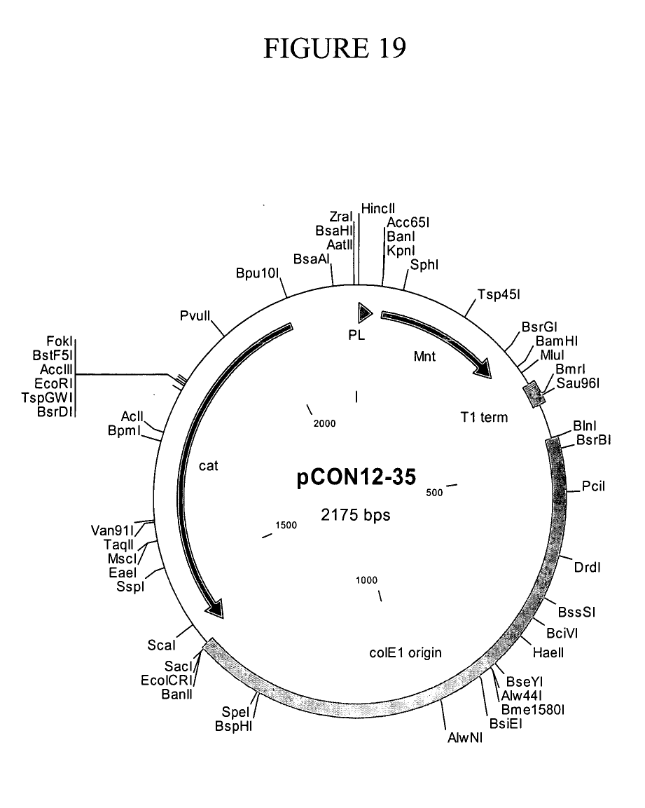

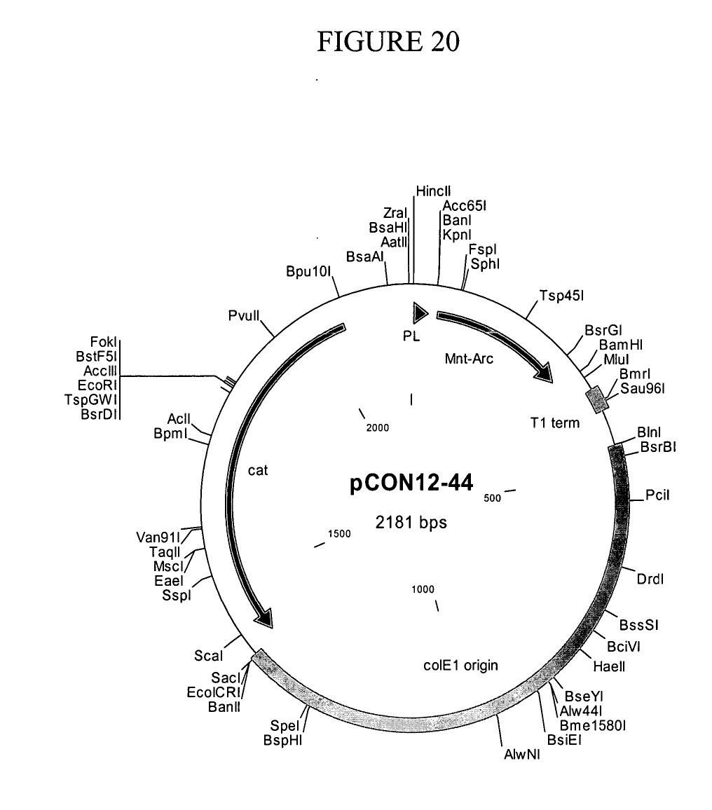

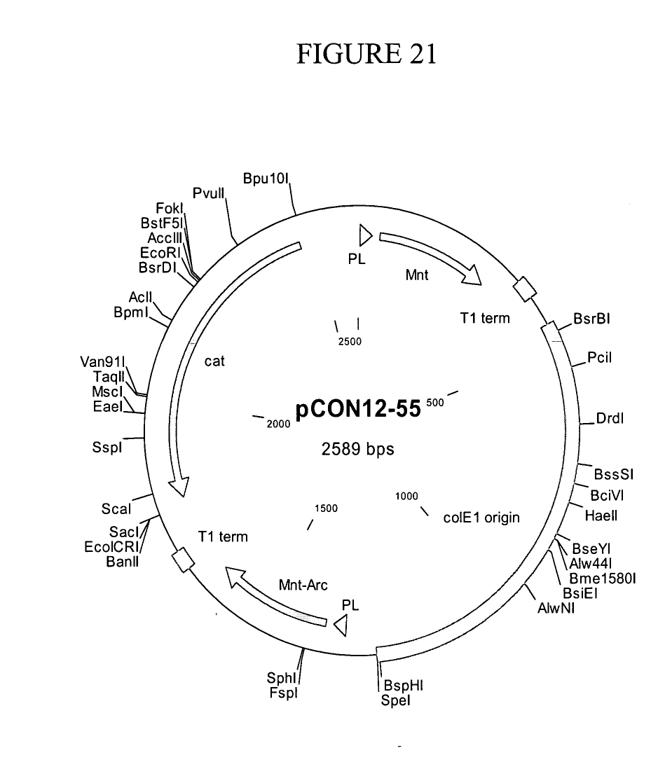

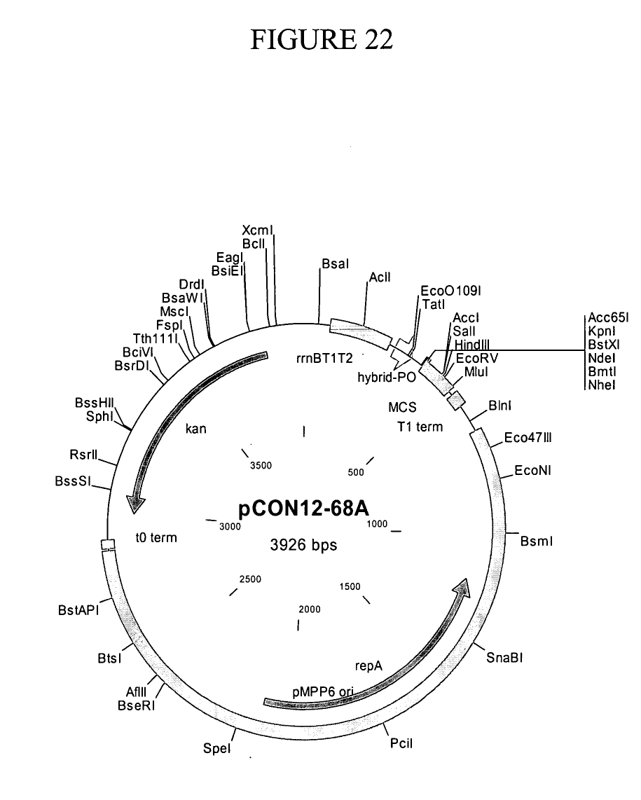

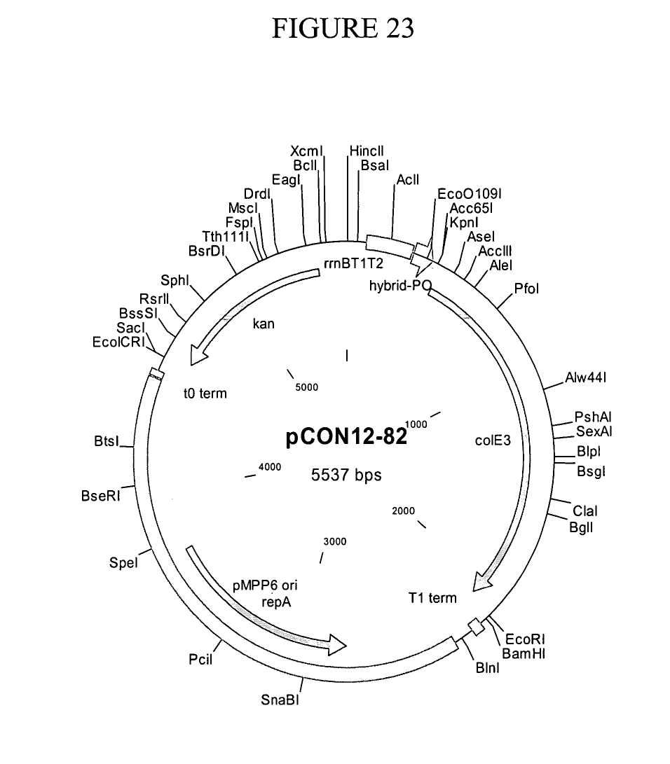

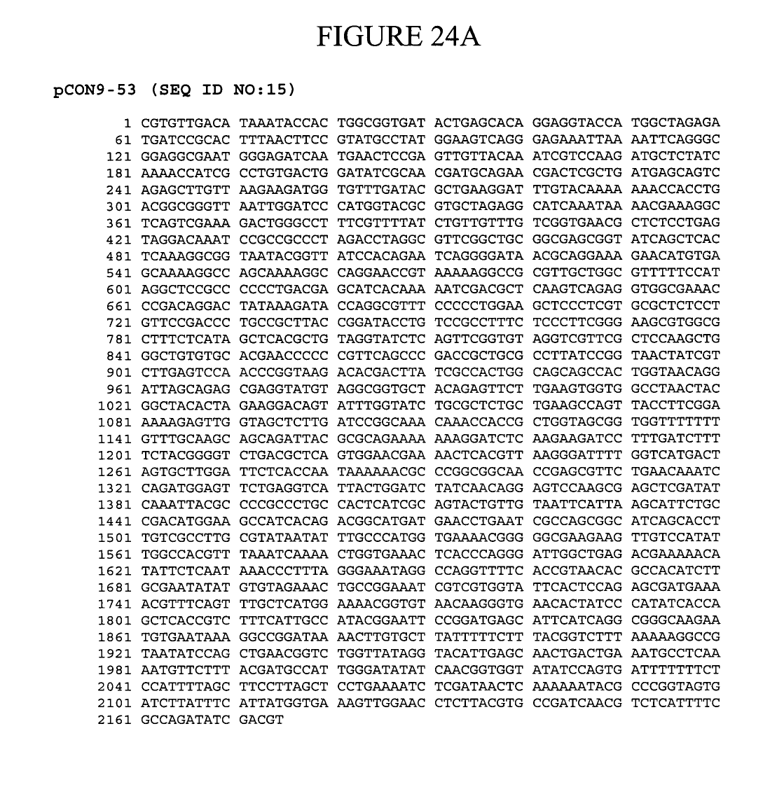

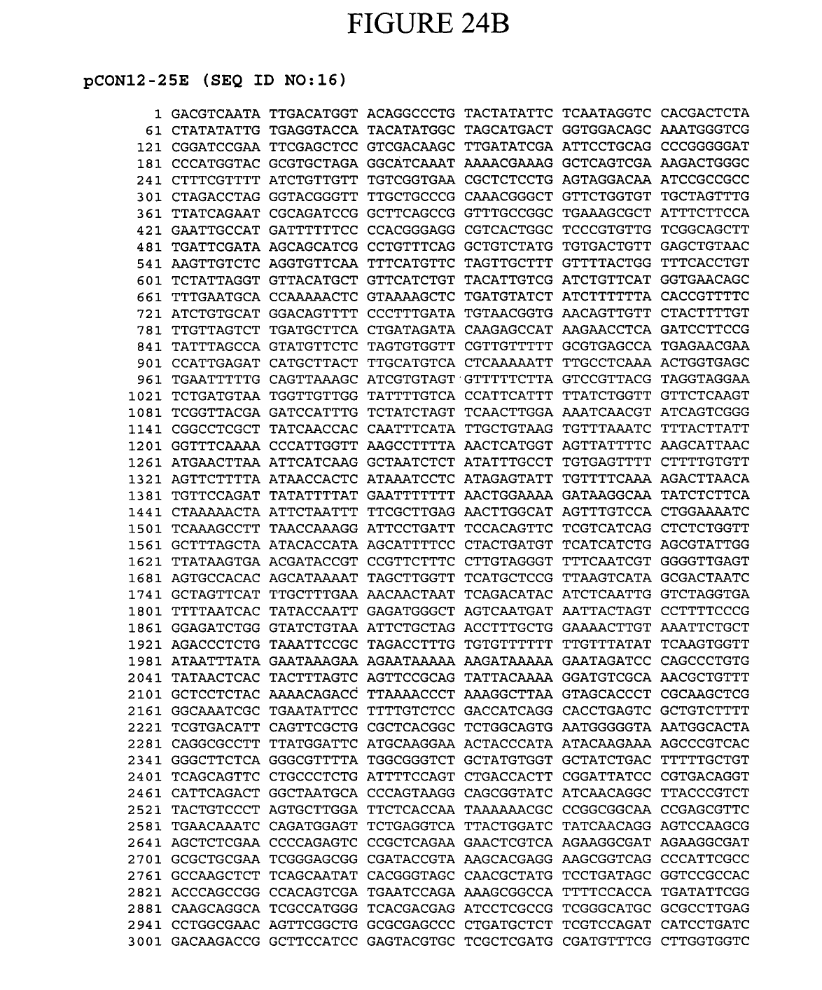

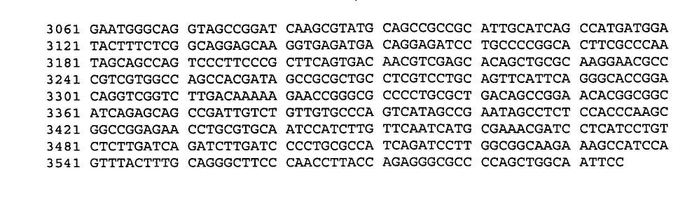

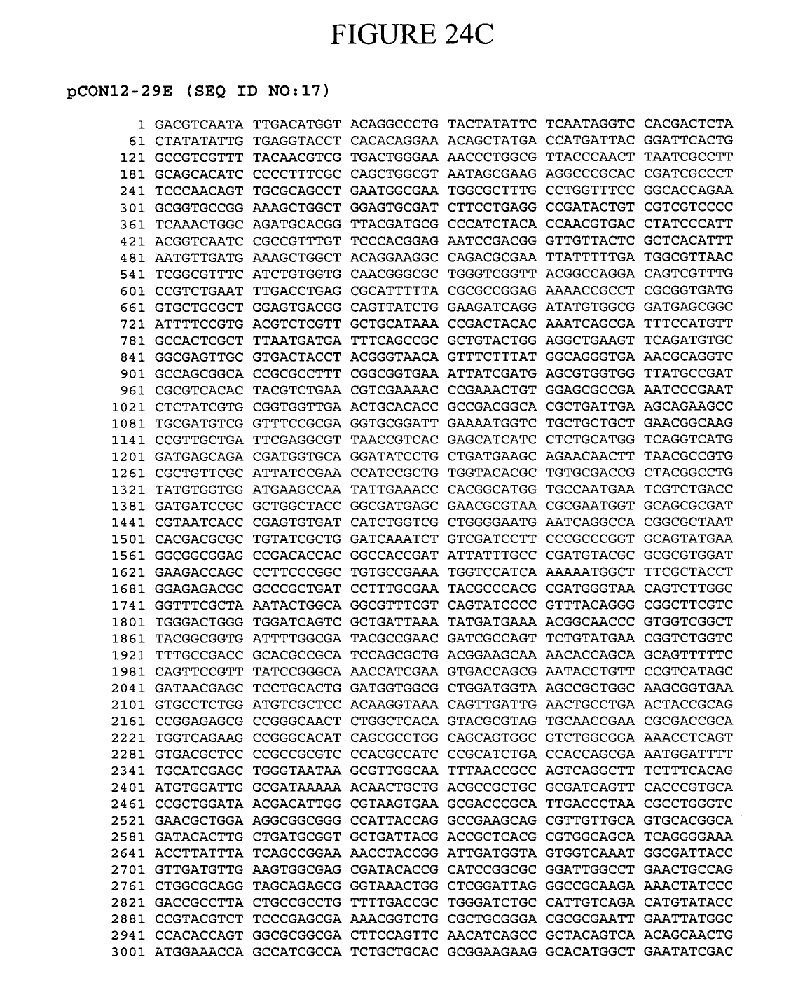

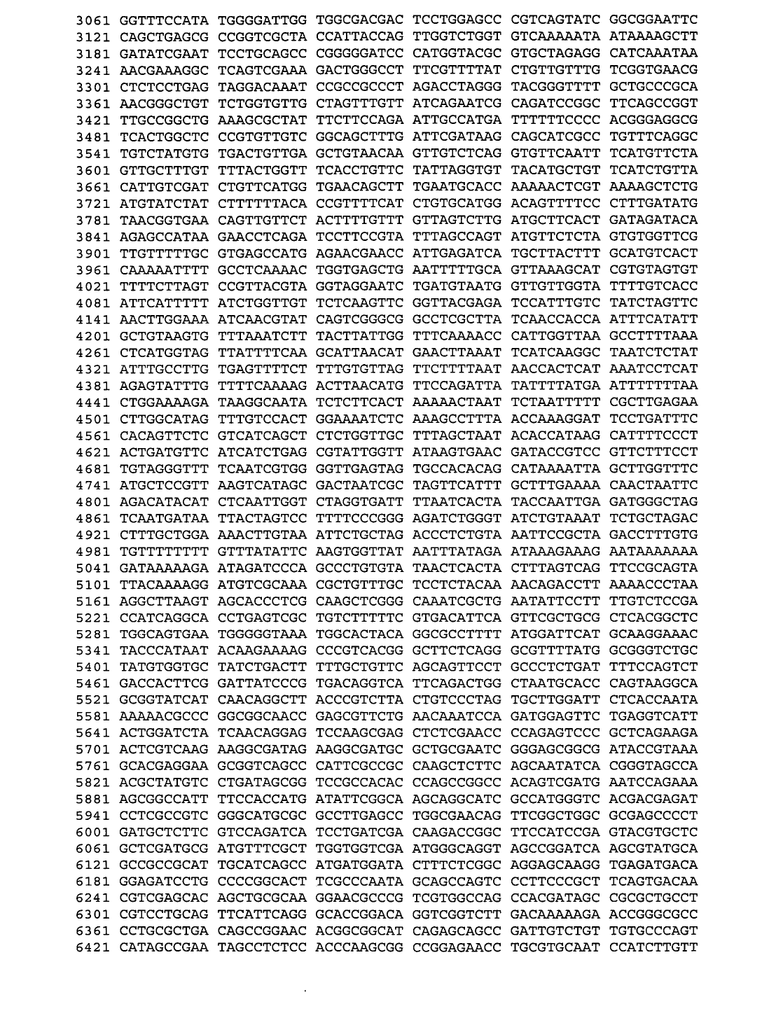

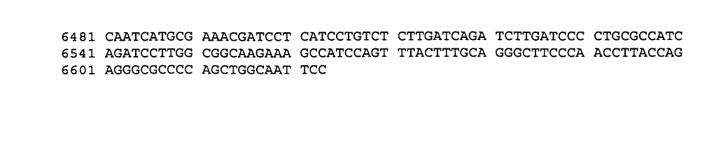

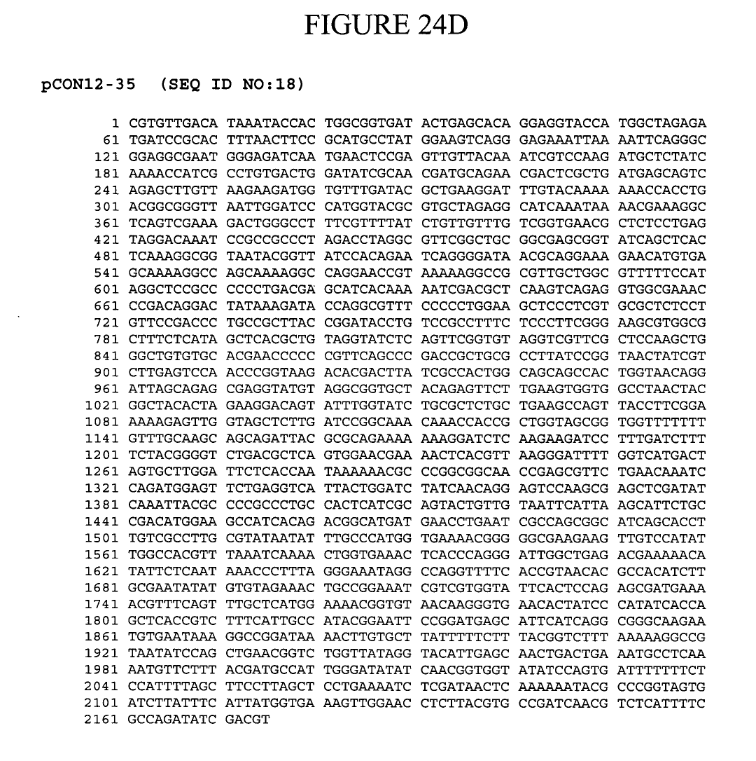

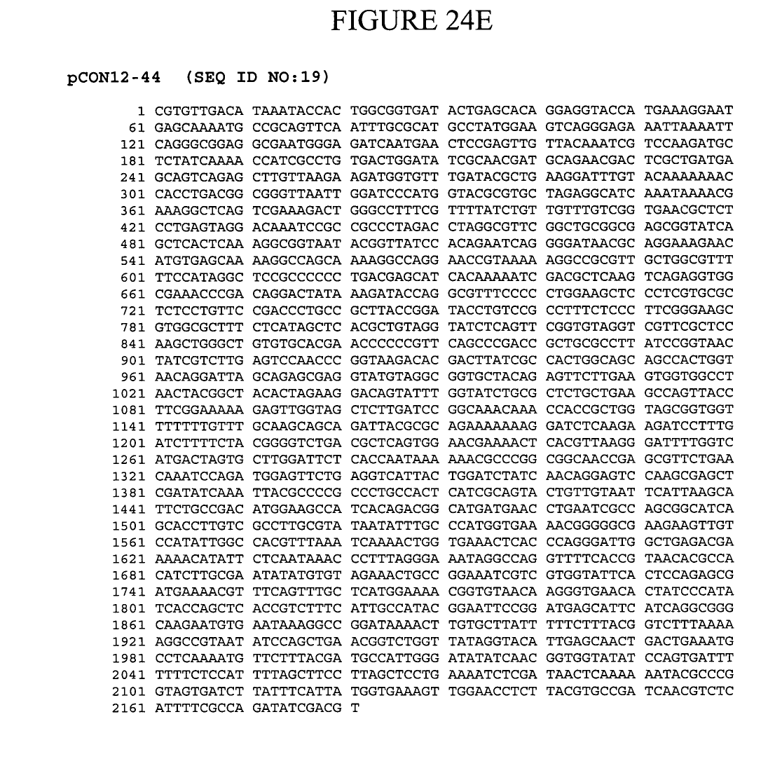

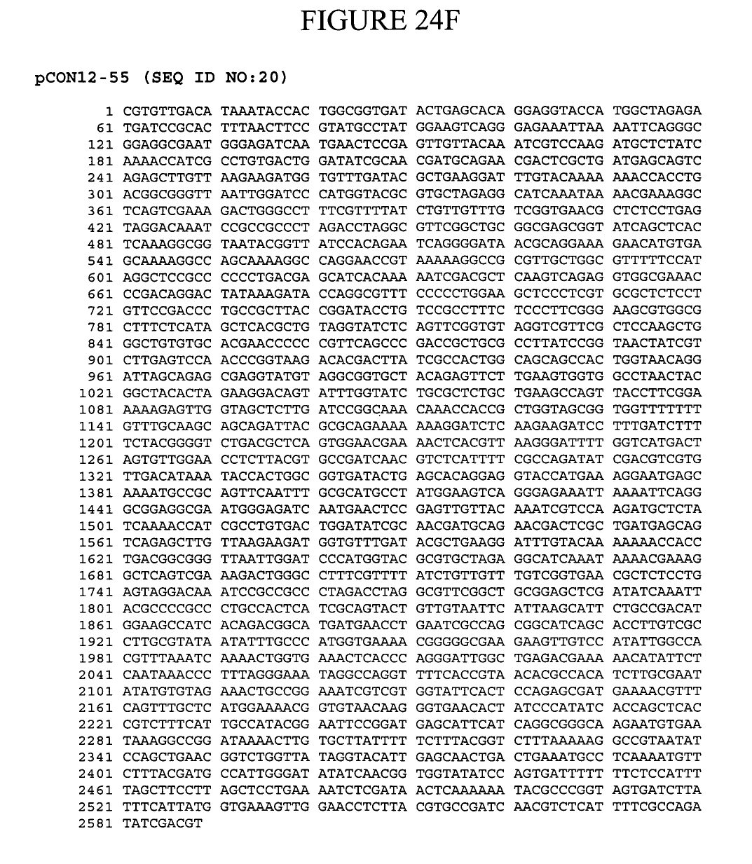

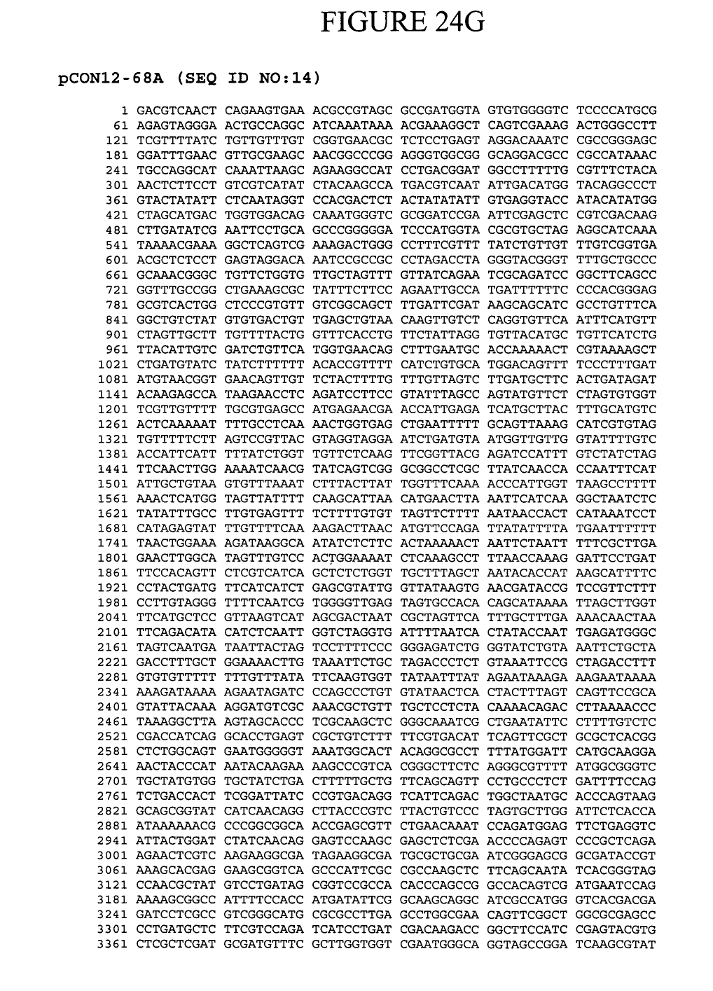

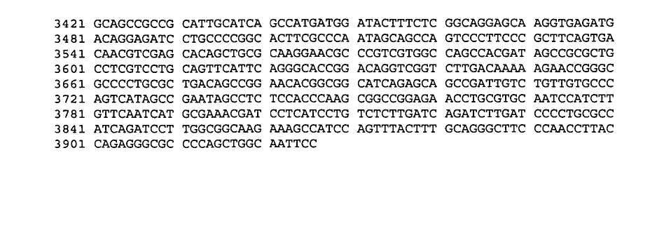

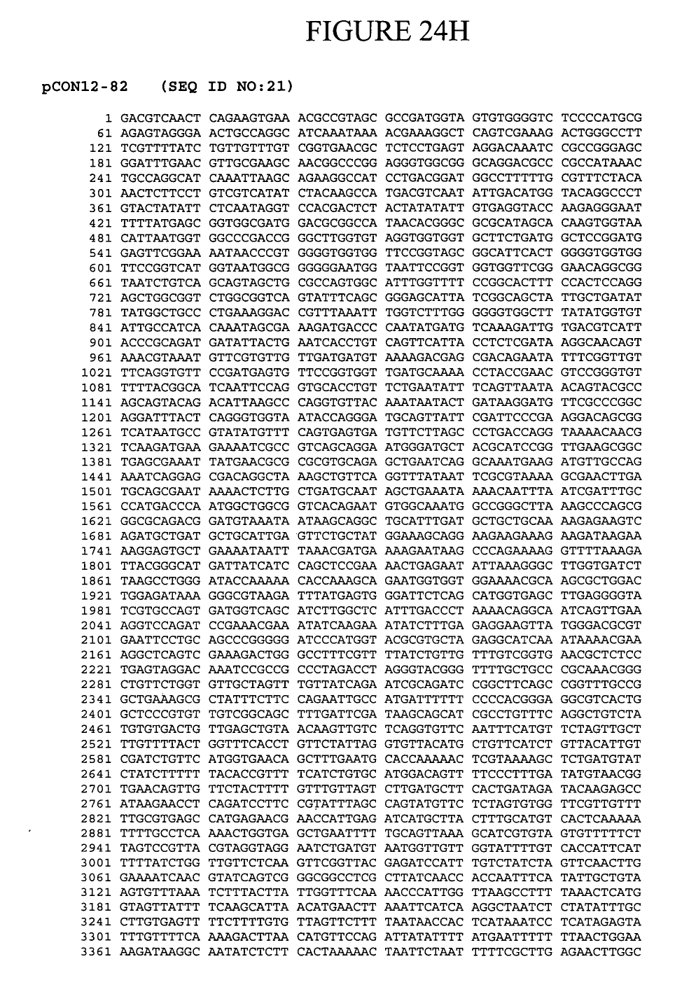

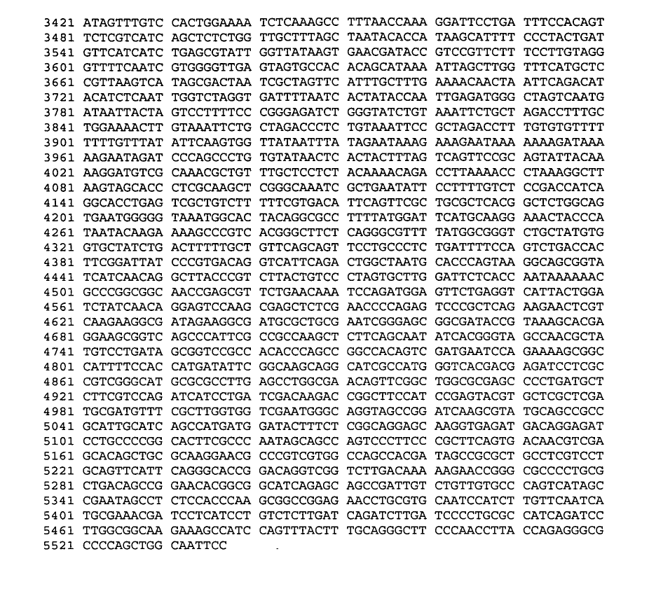

Using the standard assay for β-galactosidase activity, the promoter activities for vectors pCON12-25E and pCON12-29E in the presence and absence of repressors were obtained. Cultures were grown in Luria-Bertani broth supplemented with 50 μg/ml kanamycin (and 10 μg/ml chloramphenicol if pCON12-55 was present). Cultures were assayed in duplicate at an OD600 nm of 0.3-0.5 and expressed as Miller Units. The results are shown in FIG. 15.

As observed in FIG. 15, the promoter activities of pCON12-29E in the absence of repressor proteins (wildtype Mnt and mutant Mnt-Arc; provided by pCON12-55) were approximately 4300 Miller Units. Addition of wildtype Mnt or wildtype Arc repressors (provided on separate plasmids) to pCON12-29E did not significantly lower the level of promoter activity. However, when pCON12-29E was combined with pCON12-55, which contains both mnt and mnt-arc repressor genes, the promoter activity was reduced approximately 60-fold to a level indistinguishable from background (70 Miller Units). This assay demonstrates the tightness of the hybrid promoter/operator system for regulating gene expression.

All publications and patents mentioned in the above specification are herein incorporated by reference. Various modifications and variations of the described compositions, methods, systems, and kits of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

1. A composition comprising a vector, said vector comprising one or more transcription terminators, a promoter, a cloning site and a low copy number origin of replication, wherein said one or more transcription terminators are upstream of said promoter.

2. The composition of claim 1, wherein said transcription terminators are selected from the group of bacteriophage lambda terminators, E. coli trp gene terminators, and rrnB ribosomal terminators T1 and T2.

3. The composition of claim 2, wherein said rrnB ribosomal terminators T1 and T2 have the nucleic acid sequence of SEQ ID NO: 9.

4. The composition of claim 1, wherein said low copy number origin of replication is selected from the group consisting of a low copy number modified pSC101 origin of replication and a RK2 origin of replication.

5. The composition of claim 1, wherein said low copy number origin of replication is selected from the group consisting of a wildtype pSC101 origin of replication, a pi Sa origin of replication, and a pACYC origin of replication.

6. The composition of claim 4, wherein said low copy number modified pSC101 origin of replication has the nucleic acid sequence of SEQ ID NO:10.

7. The composition of claim 4, wherein said RK2 origin of replication has the nucleic acid sequence of SEQ ID NO:11.

8. The composition of claim 1, wherein said promoter comprises a promoter/operator.

9. The composition of claim 8, wherein said promoter/operator is the lactose promoter/operator.

10. The composition of claim 1, wherein said promoter is selected from the group consisting of PBAD, T7, and T5 promoters.

11. The composition of claim 1, wherein said promoter/operator is a hybrid mutant Mnt-Arc promoter operator.

12. The composition of claim 11, wherein said hybrid mutant Mnt-Arc promoter has the nucleic acid sequence of SEQ ID NO:13.

13. The composition of claim 1, wherein said cloning site comprises a multiple cloning site.

14. The composition of claim 1, wherein said vector further comprises a selectable marker.

15. The composition of claim 1, wherein said vector has the nucleic acid sequence of SEQ ID NO:1.

16. The composition of claim 1, wherein said vector has the nucleic acid sequence of SEQ ID NO: 2.

17. The composition of claim 1, wherein said vector has the nucleic acid sequence of SEQ ID NO: 3.

18. The composition of claim 11, wherein said vector has the nucleic acid sequence of SEQ ID NO:14.

19. The composition of claim 1, wherein said vector further comprises a nucleic acid sequence encoding a protein or RNA of interest, said nucleic acid sequence operably linked to said promoter.

20. The composition of claim 19, wherein said protein or RNA is a toxic protein or toxic RNA.

21. The composition of claim 19, wherein said protein has a toxic metabolite.

22. A composition comprising a hybrid mutant Mnt-Arc promoter nucleic acid.

23. The composition of claim 22, wherein said hybrid mutant Mnt-Arc promoter nucleic acid has the nucleic acid sequence of SEQ ID NO:13.

24. A vector comprising the nucleic acid of claim 22.

25. The vector of claim 24, wherein said vector further comprises one or more transcription terminators, a cloning site and a low copy number origin of replication, wherein said one or more transcription terminators are upstream of said promoter.

26. The vector of claim 25, wherein said transcription terminators are selected from the group of bacteriophage lambda terminators, E. coli trp gene terminators, and rrnB ribosomal terminators T1 and T2.

27. The vector of claim 26, wherein said rrnB ribosomal terminators T1 and T2 have the nucleic acid sequence of SEQ ID NO: 9.

28. The vector of claim 25, wherein said low copy number origin of replication is selected from the group consisting of a low copy number modified pSC101 origin of replication, a RK2 origin of replication, a wildtype pSC101 origin of replication, a p15a origin of replication, and a pACYC origin of replication.

29. The vector of claim 28, wherein said low copy number modified pSC101 origin of replication has the nucleic acid sequence of SEQ ID NO:10.

30. The vector of claim 28, wherein said RK2 origin of replication has the nucleic acid sequence of SEQ ID NO:11.

31. The vector of claim 25, wherein said cloning site comprises a multiple cloning site.

32. The vector of claim 25, wherein said vector further comprises a selectable marker.

33. The vector of claim 25, wherein said vector has the nucleic acid sequence of SEQ ID NO:14.

34. The vector of claim 25, wherein said vector further comprises a nucleic acid sequence encoding a protein or RNA of interest, said nucleic acid sequence operably linked to said promoter.

35. The vector of claim 34, wherein said protein or RNA is a toxic protein or toxic RNA.

36. The vector of claim 34, wherein said protein has a toxic metabolite.

37. A method, comprising:
a) providing a gene of interest in a vector, said vector comprising one or more transcription terminators, a promoter, and a low copy number origin of replication, wherein at least one of said one or more transcription terminators are upstream of said promoter and wherein said gene of interest is operably linked to said promoter; and
b) expressing said gene of interest in a bacterial host.

38. The method of claim 37, wherein said gene of interest encodes a toxic protein or RNA.

39. The method of claim 37, wherein said gene of interest encodes a protein with a toxic metabolite.

40. The method of claim 37, wherein said gene of interest is maintained in said vector under growth conditions.

41. The method of claim 40, wherein said toxic protein accumulates in said bacterial host.

42. The method of claim 37, wherein said transcription terminators are rrnB ribosomal terminators T1 and T2.

43. The method of claim 37, wherein said low copy number origin of replication is selected from the group consisting of a low copy number modified pSC101 origin of replication, a RK2 origin of replication, a wildtype pSC 101 origin of replication, a p15a origin of replication, and a pACYC origin of replication.

44. The method of claim 37, wherein said vector further comprises a promoter/operator.

45. The method of claim 37, wherein said promoter/operator is selected from the group consisting of a lactose promoter/operator and a hybrid mutant Mnt-Arc promoter operator.

46. The method of claim 45, wherein said hybrid mutant Mnt-Arc promoter has the nucleic acid sequence of SEQ ID NO:13.

47. The method of claim 37, wherein said promoter is selected from the group consisting of PBAD, T7, and T5 promoters.

48. The method of claim 37, wherein said vector further comprises a gene encoding a selectable marker.

49. The method of claim 37, wherein said vector has a nucleic acid sequence selected from the group consisting of SEQ ID NOs: 1, 2, 3, and 14.

50. The method of claim 37, wherein said bacterial host is a gram negative bacteria.

51. The method of claim 50, wherein said gram negative bacteria is E. coli.

52. A method, comprising:
a) providing a gene of interest in a vector, said vector comprising a hybrid mutant Mnt-Arc promoter nucleic acid, wherein said gene of interest is operably linked to said promoter; and
b) expressing said gene of interest in a bacterial host.

53. The method of claim 52, wherein said gene of interest encodes a toxic protein or RNA.

54. The method of claim 52, wherein said gene of interest encodes a protein with a toxic metabolite.

55. The method of claim 52, wherein said gene of interest is maintained in said vector under growth conditions.

56. The method of claim 55, wherein said toxic protein accumulates in said bacterial host.

57. The method of claim 52, wherein said vector further comprises one or more transcription terminators and a low copy number origin of replication, wherein at least one of said one or more transcription terminators are upstream of said promoter operator.

58. The method of claim 57, wherein said transcription terminators are rrnB ribosomal terminators T1 and T2.

59. The method of claim 57, wherein said low copy number origin of replication is selected from the group consisting of a low copy number modified pSC101 origin of replication, a RK2 origin of replication, a wildtype pSC101 origin of replication, a p15a origin of replication, and a pACYC origin of replication.

60. The method of claim 52, wherein said vector further comprises a gene encoding a selectable marker.

61. The method of claim 52, wherein said vector has a nucleic acid sequence selected from the group consisting of SEQ ID NOs: 1, 2, 3, and 14.

62. The method of claim 52, wherein said bacterial host is a gram negative bacteria.

63. The method of claim 62, wherein said gram negative bacteria is E. coli.

64. The method of claim 52, further comprising providing a hybrid mutant Mnt-Arc repressor protein.

65. A kit, comprising
a) vector comprising a hybrid mutant Mnt-Arc promoter nucleic acid; and
b) a hybrid mutant Mnt-Arc repressor protein.

66. The kit of claim 65, wherein said a hybrid mutant Mnt-Arc promoter nucleic acid has the nucleic acid sequence of SEQ ID NO:13.

67. The kit of claim 65, further comprising instructions for using said kit for expressing a gene of interest encoding a toxic protein or RNA.
